SEQUENCE LISTING (1 sequence)

SEQ ID NO: 1 - protein, 9 aa, Human papillomavirus
LQSSLHLTA

(19) United States
(12) Patent Application Publication
(10) Pub. No.: US 2023/0138309 A1
(43) Pub. Date: May 4, 2023

(54) METHODS OF ISOLATING T-CELLS AND T-CELL RECEPTORS FROM TUMOR BY SINGLE-CELL ANALYSIS FOR IMMUNOTHERAPY

(71) Applicant: The United States of America,as represented by the Secretary,Department of Health and Human Services, Bethesda, MD (US)

(72) Inventors: Sri Krishna, North Bethesda, MD (US); Frank J. Lowery, III, Clarksburg, MD (US); Ken-ichi Hanada, Bethesda, MD (US); James C. Yang, Bethesda, MD (US); Steven A. Rosenberg, Potomac, MD (US); Paul F. Robbins, Chevy Chase, MD (US); Rami Yoseph, Gaithersburg, MD (US)

(73) Assignee: The United States of America,as represented by the Secretary,Department of Health and Human Services, Bethesda, MD (US)

(21) Appl. No.: 17/912,315

(22) PCT Filed: Mar. 19, 2021
(86) PCT No.: PCT/US2021/023240

§ 371 Date: Sep. 16, 2022

Related U.S. Application Data
(60) Provisional application No. 62/992,701, filed on Mar. 20, 2020.

(51) Int. Cl. C12N 15/10; C12N 5/0783; C12Q 1/6881

(52) U.S. Cl. CPC C12N 15/1072; C12N 5/0636; C12Q 1/6881; C12Q 1/6869

(57) ABSTRACT

Provided are methods of preparing an enriched population of T cells having antigenic specificity for a target antigen. The method may comprise isolating T cells from a tumor sample of a patient; selecting the isolated T cells which have a gene expression profile; and separating the selected T cells from the unselected cells. The separated selected T cells provide an enriched population of T cells having antigenic specificity for the target antigen. Methods of isolating a T cell receptor (TCR), preparing a population of cells that express a TCR, isolated TCRs, isolated populations of cells, pharmaceutical compositions, and methods of treating or preventing a condition in a mammal are also provided.

### CROSS-REFERENCE TO RELATED APPLICATIONS

This patent application claims the benefit of U.S. Provisional Patent Application No. 62/992,701, filed Mar. 20, 2020, which is incorporated by reference in its entirety herein.

### STATEMENT REGARDING FEDERALLY SPONSORED RESEARCH OR DEVELOPMENT

This invention was made with Government support under project number ZIA BC 010984 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 553 Byte ASCII (Text) file named “753067_ST25.TXT,” dated Mar. 18, 2021.

### BACKGROUND OF THE INVENTION

Adoptive cell therapy (ACT) using T cells that target a neoantigen encoded by a cancer-specific mutation can produce positive clinical responses in some patients. Nevertheless, several obstacles to the successful use of ACT for the treatment of cancer and other conditions remain. For example, the current methods used to produce cancer-reactive T cells require significant time and may not readily identify the desired T cell receptors that bind cancer targets. Accordingly, there is a need for improved methods of obtaining an isolated population of cells for ACT.

### BRIEF SUMMARY OF THE INVENTION

An aspect of the invention provides a method of preparing an enriched population of T cells having antigenic specificity for a target antigen, the method comprising: isolating T cells from a tumor sample of a patient; selecting the isolated T cells which have a gene expression profile; separating the selected T cells from the unselected cells, wherein the separated selected T cells provide an enriched population of T cells having antigenic specificity for the target antigen, wherein the target antigen is a neoantigen encoded by a cancer-specific mutation, a cancer antigen, or a cancer-associated viral antigen, and the gene expression profile comprises: (a) (i) one or both of CD4⁺ and CD8⁺ and (ii) one or more of AFAP1IL2⁺, ASB2⁺, CXCL13⁺, HMOX1⁺, ITM2A⁺, KLRB1⁺, PDLIM4⁺, TIGIT⁺, LTB⁻, LYAR⁻, RGCC⁻, and S100A10⁻; (b) CD4⁺ and one or more of BATF⁺, CD247⁺, CXCL13⁺, DNPH1⁺, DUSP4⁺, GYPC⁺, IGFLR1⁺, ITM2A⁺, KLRB1⁺, LIMS1⁺, NMB⁺, NR3C1⁺, SH2D1A⁺, SPOCK2⁺, SUPT3H⁺, TIGIT⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻; (c) CD8⁺ and one or more of AFAP1IL2⁺, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA1⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, TGIT⁺, ANXA1⁻, EEF1B2⁻, EMP3⁻, IL7R⁻, LGALS3⁻, LTB⁻, LYAR⁻, RGCC⁻, RPL36A⁻, and S100A10⁻; (d) CD8⁺ and one or more of CD39⁺, CD74⁺, CD103⁺, CD106⁺, CD137⁺, HLA-DR⁺, TIGIT⁺, CCR7⁻, CD8A⁻, CD16⁻, CD45RA⁻, CD62L⁻ and IL7R⁻; (e) one or more of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺, SMC4⁺, SUPT3H⁺, TIGIT⁺, TNFRSF18⁺, TOX⁺, TRAF3IP3⁺, and YPEL2⁺; (f) CD4⁺ and one or more of ADI1⁺, AHI1⁺, ARID5B⁺, BATF⁺, CMTM7⁺, CPM⁺, CXCL13⁺, CYTH1⁺, ELMO1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TIGIT⁺, TLK1⁺, TMEM123⁺, TMEM70⁺, TNIK⁺, TOX⁺, TSHZ2⁺, UCP2⁺, VOPP1⁺, and YPEL2⁺; (g) CD8⁺ and one or more of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺; (h) AHI1⁺, CXCL13⁺, FABP5⁺, NAP1L4⁺, ORMDL3⁺, PPP1R16B⁺, SH2D1A⁺, TIGIT⁺, and TOX⁺; or (i) one or more of TIGIT⁺, CD39⁺, and PD-1⁺.

Another aspect of the invention provides a method of isolating a T cell receptor (TCR), or an antigen-binding portion thereof, having antigenic specificity for a target antigen, the method comprising: preparing an enriched population of T cells having antigenic specificity for the target antigen according to any of the methods described herein with respect to other aspects of the invention; sorting the T cells in the enriched population into separate single T cell samples; sequencing TCR complementarity determining regions 3 (CDR3) in one or more of the separate single T cell samples; pairing an alpha chain variable region comprising a CDR3 with a beta chain variable region comprising a CDR3 encoded by the nucleic acid of the separate single T cell samples; introducing a nucleotide sequence encoding the paired alpha chain variable region and beta chain variable region into host cells and expressing the paired alpha chain variable region and beta chain variable region by the host cells; screening the host cells expressing the paired alpha chain variable region and beta chain variable region for antigenic specificity for the target antigen; and selecting the paired alpha chain variable region and beta chain variable region that have antigenic specificity for the target antigen, wherein the TCR, or an antigen-binding portion thereof, having antigenic specificity for the target antigen is isolated.

Another aspect of the invention provides a method of preparing a pooled population of cells that express a TCR, or an antigen-binding portion thereof, having antigenic specificity for a target antigen, the method comprising: (a) preparing an enriched population of T cells having antigenic specificity for the target antigen according to any of the methods described herein with respect to other aspects of the invention; (b) sorting the T cells in the enriched population into separate single T cell samples; (c) sequencing TCR CDR3 in the separate single T cell samples; (d) pairing an alpha chain variable region comprising a CDR3 with a beta chain variable region comprising a CDR3 encoded by the nucleic acid of the separate single T cell samples; (e) introducing a nucleotide sequence encoding the paired alpha chain variable region and beta chain variable region into PBMC and expressing the paired alpha chain variable region and beta chain variable region by the PBMC; and (f) carrying out (c), (d), and (e) for a plurality of the separate single T cell samples of the enriched population of T cells having antigenic specificity for the target antigen prepared according to (a), thereby providing a pooled population of cells that express a TCR, or an antigen-binding portion thereof, having antigenic specificity for a target antigen.

Another aspect of the invention provides a method of isolating a TCR, or an antigen-binding portion thereof, having antigenic specificity for a target antigen, the method comprising: isolating T cells from a tumor sample of a patient; sorting the T cells in the enriched population into separate single T cell samples; sequencing TCR CDR3 in the separate single T cell samples; selecting the separate single T cell samples which have a gene expression profile; pairing an alpha chain variable region comprising a CDR3 with a beta chain variable region comprising a CDR3 encoded by the nucleic acid of the separate single T cell samples with the gene expression profile; introducing a nucleotide sequence encoding the paired alpha chain variable region and beta chain variable region into host cells and expressing the paired alpha chain variable region and beta chain variable region by the host cells; screening the host cells expressing the paired alpha chain variable region and beta chain variable region for antigenic specificity for the target antigen; and selecting the paired alpha chain variable region and beta chain variable region that have antigenic specificity for the target antigen, wherein the TCR, or an antigen-binding portion thereof, having antigenic specificity for the target antigen is isolated, wherein the gene expression profile comprises: (a) (i) one or both of CD4⁺ and CD8⁺ and (ii) one or more of AFAP1IL2⁺, ASB2⁺, CXCL13⁺, HMOX1⁺, ITM2A⁺, KLRB1⁺, PDLIM4⁺, LTB⁻, LYAR⁻, RGCC⁻, and S100A10⁻; (b) CD4⁺ and one or more of BATF⁺, CD247⁺, CXCL13⁺, DNPH1⁺, DUSP4⁺, GYPC⁺, IFITM1⁺, IGFLR1⁺, ITM2A⁺, KLRB1⁺, LIMS1⁺, NMB⁺, NR3C1⁺, SH2D1A⁺, SPOCK2⁺, SUPT3H⁺, TIGIT⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻; (c) CD8⁺ and one or more of AFAP1IL2⁺, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA1⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, TIGIT⁺, ANXA1⁻, EEF1B2⁻, EMP3⁻, IL7R⁻, LGALS3⁻, LTB⁻, LYAR⁻, RGCC⁻, RPL36A⁻, and S100A10⁻; (d) CD8+ and one or more of CD39⁺, CD74⁺, CD103⁺, CD106⁺, CD137⁺, HLA-DR⁺, TIGIT⁺, CCR7⁻, CD8A⁻, CD16⁻, CD45RA⁻, CD62L⁻ and IL7R⁻; (e) one or more of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺, SMC4⁺, SUPT3H⁺, TIGIT⁺, TNFRSF18⁺, TOX⁺, TRAF3IP3⁺, and YPEL2⁺; (f) CD4⁺ and one or more of ADI1⁺, AHI1⁺, ARID5B⁺, BATF⁺, CMTM7⁺, CPM⁺, CXCL13⁺, CYTH1⁺, ELMO1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TIGIT⁺, TLK1⁺, TMEM123⁺, TMEM70⁺, TNIK⁺, TOX⁺, TSHZ2⁺, UCP2⁺, VOPP1⁺, and YPEL2⁺; (g) CD8⁺ and one or more of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺; (h) one or more of AHI1⁺, CXCL13⁺, FABP5⁺, NAP1L4⁺, ORMDL3⁺, PPP1R16B⁺, SH2D1A⁺, TIGIT⁺, and TOX⁺; or (i) one or more of TIGIT⁺, CD39⁺, and PD-1⁺.

Still another aspect of the invention provides a method of preparing a population of cells that express a TCR, or an antigen-binding portion thereof, having antigenic specificity for a target antigen, the method comprising: isolating a TCR, or an antigen-binding portion thereof, according to any of the methods described herein with respect to other aspects of the invention, and introducing a nucleotide sequence encoding the isolated TCR, or the antigen-binding portion thereof, into peripheral blood mononuclear cells (PBMC) to obtain cells that express the TCR, or the antigen-binding portion thereof

Further aspects of the invention provide related TCRs, or antigen-binding portions thereof, isolated populations of cells, and pharmaceutical compositions prepared according to any of the inventive methods.

Additional aspects of the invention provide related methods of treating or preventing a condition in a mammal and related methods of preparing a medicament for the treatment or prevention of the condition in a mammal, wherein the condition is cancer or a viral condition.

### BRIEF DESCRIPTION OF THE SEVERAL VIEWS OF THE DRAWING(S)

FIG. 1A shows the results of the t-SNE analysis of T cells from colorectal cancer Patient 4323 (t-SNE map). The clusters are numbered 0-7.

FIG. 1B shows the known neoantigen-reactive TCRs projected onto the t-SNE map of FIG. 1A. The known neoantigen-reactive TCRs localized to cluster 5 (boxed area).

FIG. 1C shows the expression of selected genes by 4323 T cells in cluster 5 of FIG. 1A.

FIG. 2A is a t-SNE map for the TIL of Patient 4323 showing that all neoantigen-reactive TCRs that were prospectively re-constructed based on the cluster transcriptome profile were located in cluster 5 (boxed area).

FIG. 2B is a t-SNE map for the TIL of Patient 4323 showing that all of the non-reactive TCRs tested were located in all eight clusters (dark circles) indicating specificity.

FIG. 3A shows the results of the t-SNE analysis of T cells from colorectal cancer Patient 4324 (t-SNE map). The clusters are numbered 0-6.

FIG. 3B shows the known neoantigen-reactive TCRs projected onto the t-SNE map of FIG. 3A. The known neoantigen-reactive TCRs localized to cluster 6 (boxed area).

FIG. 3C shows the expression of selected genes by 4324 T cells in cluster 6 of FIG. 3A.

FIG. 4A shows the results of the t-SNE analysis of T cells from breast cancer Patient 4322 (t-SNE map). The clusters are numbered 0-8.

FIG. 4B shows the known neoantigen-reactive TCRs projected onto the t-SNE map of FIG. 4A. The known neoantigen-reactive TCRs localized to cluster 3 (boxed area).

FIG. 4C shows the expression of selected genes by 4322 T cells in cluster 3 of FIG. 4A.

FIG. 5A shows the results of the combined t-SNE analysis of CD8⁺ T cells from previous colorectal cancer patient 4323 and lung cancer Patients 4234 and 4237 (t-SNE map). The clusters are numbered 0-6.

FIG. 5B shows the known neoantigen-reactive TCRs projected onto the t-SNE map of FIG. 5A and the re-clustering of 4323 CD8⁺ clusters with 4234 and 4237. The known neoantigen-reactive TCRs localized to cluster 4 (boxed area).

FIG. 5C shows the expression of selected genes by CD8⁺ 4323, 4234, and 4237 T cells in cluster 4 of FIG. 5A.

FIG. 6 is a graph showing the NeoTCR Signature Score for the neoantigen-reactive T cells of Patient 4323 (n=236 cells) and the cells other than the neoantigen-reactive T cells of Patient 4323 (n=2597).

FIG. 7A shows the results of the t-SNE analysis of T cells from colorectal cancer Patient 4283 (t-SNE map). The clusters are numbered 0-4.

FIG. 7B shows the known neoantigen-reactive TCRs projected onto the t-SNE map of FIG. 7A. The known CD4⁺ neoantigen-reactive TCRs localized to cluster 2 (boxed area).

FIG. 7C shows the expression of selected genes by 4283 T cells in cluster 2 of FIG. 7A.

FIG. 8A shows the cells expressing the 95th percentile of NeoTCR signature derived from the NeoTCR cluster transcriptome profile of Pt.4323 (darker dots) projected onto the original tSNE plots of other patients.

FIG. 8B shows the cells expressing the 95th percentile of NeoTCR signature derived from Pt.4322 (darker dots) projected onto the original tSNE plots of other patients.

FIG. 8C shows the cells expressing the 95th percentile of NeoTCR signature derived from Pts. 4323, 4234, and 4237 (darker dots) projected onto the original tSNE plots of other patients.

FIG. 9 shows plots comparing the clustering of T cells analyzed by antibody-based tSNE and transcriptome-based tSNE. The T cells were reactive against six neoantigens (DOPEY2, U2AF1, SLFN11, BPNT1, and MLLT4) from three NSCLC patients (4234, 4237, and 4369). Neoantigen-reactive CD8⁺ T-cells are represented by darker dots.

FIG. 10 shows tSNE plots for Patient 4234. The two tSNE plots in the box show the distribution of CD8⁺ cells and the neoantigen-reactive CD8+ T-cells in the TIL of Patient 4234, respectively. The ten tSNE plots outside the box show the distributions of cells that express the indicated molecules associated with neoantigen-reactive T-cells. Results for a representative ten molecules are shown, and in all of the plots, dark dots represent the cells associated with the feature indicated above each plot.

FIGS. 11A-11D show the expression of cell surface proteins as detected by FBC antibodies. Black dots represent neoantigen-reactive T-cells and gray dots represent other, non-antigen-reactive T-cells in the TIL of Patient 4234. FIG. 11A: CD8A expression is low (dim) on neoantigen-reactive T-cells. FIG. 11B: Both CCR7 and CD45RA expressions are low, suggesting that neoantigen-reactive cells are effector memory T-cells. FIG. 11C: Neoantigen-reactive cells have low (dim positive) CD103 expression and are CD39 positive. FIG. 11D: The majority of neoantigen-reactive CD8 T-cells express both PD-1 and Tim-3.

FIG. 12 is a schematic illustrating a workflow for rapid neo-antigen TCR isolation from tumors using single cell analysis according to aspects of the invention. Aspects of the invention may provide, for example, two ways of obtaining anti-tumor mutation-specific neoantigen reactive TCRs for immunotherapy: (1) Single cell RNA sequencing and subsequent application of NeoTCR gene signature to in silico reconstruct the TCRs and (2) direct isolation of tumor neoantigen-reactive TCRs by flow cytometry based sorting using minimal markers followed by TCR reconstruction.

FIG. 13 presents FACS data showing 4-1BB expression by effector cells transduced with 4397 TCR1 following co-culture with target cells treated with DMSO (control) (left panel) or target cells presenting HPV16 E4 (right panel).

### DETAILED DESCRIPTION OF THE INVENTION

While many tumors may contain tumor-infiltrating lymphocytes (TILs), only a fraction of these may be actually reactive with cancer mutation-encoded neoantigens. Many of the TILs resident within a given tumor may be bystander T cells that do not directly participate in a targeted immune rejection of the tumor. Previous efforts to identify markers that enrich the tumor-targeting T cells out of a mixed population have achieved varying success and little consensus. Previous efforts to treat patients with TIL fragment cultures selected on the basis of in vitro neoantigen reactivity have shown the ability of TIL to mediate long-term regressions in patients with advanced metastatic cancer. However, TIL fragment screening may be a slow and labor-intensive process that may not result in the ability to treat patients with pure tumor-reactive TIL populations. Rather, TIL fragment screening may only select the TIL fragments with the highest degree of in vitro reactivity for expansion. Such techniques may be a stochastic process in which tumor-reactive TIL may be outgrown by tumor-irrelevant competitors, resulting in a treatment product of diminished reactivity. The search for markers of autologous tumor-reactive T-cells has shown that some markers, such as PD-1 and CD39, can enrich for tumor-reactive T cells, but it is not clear that such enrichment is sufficient to allow the identification of TCR sequences which could be applied to engineering T-cell therapies. Similar challenges exist with respect to the identification of T cells reactive to cancer-associated viral antigens.

The inventive methods may ameliorate these and other disadvantages by rapidly identifying TCR sequences of T-cells reactive against antigens, e.g., cancer-specific antigens and cancer-associated viral antigens which could be used to engineer T-cells for therapy. The inventive methods may, advantageously, avoid the uncertainties associated with finding, growing and administering native TIL populations containing lower frequencies of such cells.

It has been discovered that single-cell analysis of T cells isolated from tumor specimens has revealed a cell population present in multiple common epithelial cancers that encompass the majority of previously identified TCRs reactive against target antigens. This population may be defined by the gene expression profiles described herein. Using, for example, clonally defined T-cell receptors targeting unique somatic personalized mutations from a patient's tumor, new unknown TCRs expressed by cells with the gene expression profiles described herein were reconstructed and were found to be cancer neoantigen-reactive. Aspects of the invention also provide an independent method using CITE-seq analysis of the gene expression profiles that selects and identifies cancer neoantigen-reactive T-cells. The inventive methods dramatically increase the potential to rapidly isolate T cells and TCRs for cell-based immunotherapies of common cancers without the need for growing tumor infiltrating T-cells and expensive and time-consuming screening. The gene expression profiles described herein may also, advantageously, identify T cells and TCRs reactive to cancer-associated viral antigens.

It has also been discovered that there exists a well-defined population of cancer neoantigen-reactive TIL in tumors of multiple histologies and that this population's signature is robust enough to prospectively identify cancer neoantigen-reactive TIL out of a mixed population. Utilizing gene expression profiles identified by the inventive methods described herein, it is possible to accurately analyze single T-cells from tumor and use the TCR information to prospectively synthesize cancer neoantigen reactive TCRs for patient treatment.

An aspect of the invention provides a method of preparing an enriched population of T cells having antigenic specificity for a target antigen. The phrases “antigen-specific” and “antigenic specificity,” as used herein, mean that the T cell can specifically bind to and immunologically recognize an antigen, or an epitope thereof, such that binding of the T cell to the antigen, or the epitope thereof, elicits an immune response. In this regard, the T cell populations obtained by the inventive methods may comprise a higher proportion of T cells having antigenic specificity for a target antigen as compared to cell populations that have not been obtained by the inventive methods.

In an aspect of the invention, the target antigen is a cancer antigen. The term “cancer antigen,” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen can additionally be expressed by cells of a different state of development or maturation. For instance, the cancer antigen can be additionally expressed by cells of the embryonic or fetal stage, which cells are not normally found in an adult host. Alternatively, the cancer antigen can be additionally expressed by stem cells or precursor cells, which cells are not normally found in an adult host. Cancer antigens are known in the art and include, for instance, mesothelin, CD19, CD22, CD276 (B7H3), gp100, MART-1, Epidermal Growth Factor Receptor Variant III (EGFRVIII), TRP-1, TRP-2, tyrosinase, NY-ESO-1 (also known as CAG-3), MAGE-1, MAGE-3, etc.

In an aspect of the invention, the target antigen is a neoantigen encoded by a cancer-specific mutation. Neoantigens are a class of cancer antigens which arise from cancer-specific mutations in expressed protein. The term “neoantigen” relates to a peptide or protein expressed by a cancer cell that includes one or more amino acid modifications compared to the corresponding wild-type (non-mutated) peptide or protein that is expressed by a normal (non-cancerous) cell. A neoantigen may be patient-specific. A “cancer-specific mutation” is a somatic mutation that is present in the nucleic acid of a tumor or cancer cell but absent in the nucleic acid of a corresponding normal, i.e. non-tumorous or non-cancerous, cell.

In an aspect of the invention, the target antigen is a viral-specific antigen. Viral-specific antigens are known in the art and include, for example, any viral protein or peptide expressed or presented by virally-infected cells (APCs) which are not expressed or presented by cells which are not infected by a virus, e.g., env, gag, pol, gp120, thymidine kinase, and the like. In an aspect of the invention, the viral-specific antigen is a cancer-associated viral antigen, for example, human papillomavirus (HPV) 16 E4, HPV 16 E6, HPV 16 E7, HPV 18 E6, HPV 18 E7, and the like. The viral-specific antigen may be, for example, a herpes virus antigen, pox virus antigen, hepadnavirus antigen, papilloma virus antigen, adenovirus antigen, coronavirus antigen, orthomyxovirus antigen, paramyxovirus antigen, flavivirus antigen, and calicivirus antigen. For example, the viral-specific antigen may be selected from the group consisting of respiratory syncytial virus (RSV) antigen, influenza virus antigen, herpes simplex virus antigen, Epstein-Barr (EBV) virus antigen, HPV antigen, varicella virus antigen, cytomegalovirus antigen, hepatitis A virus antigen, hepatitis B virus antigen, hepatitis C virus antigen, human immunodeficiency virus (HIV) antigen, human T-lymphotropic virus antigen, calicivirus antigen, adenovirus antigen, and Arena virus antigen. In an aspect of the invention, the cancer-associated viral antigen is a HPV antigen.

The method may comprise isolating T cells from a tumor sample of a patient. The tumor sample may be, for example, tissue from primary tumors or tissue from the site of metastatic tumors. As such, the tumor sample may be obtained by any suitable means, including, without limitation, aspiration, biopsy, or resection. In an aspect of the invention, the patient is a cancer patient. In another aspect of the invention, the patient is a patient suffering from a viral condition.

The method may further comprise selecting the isolated T cells which have a gene expression profile. Selecting the isolated T cells which have the gene expression profile may comprise sorting the T cells into separate single T cell samples and separately detecting the expression and/or non-expression of one or more genes by one or more single T cells. In an aspect of the invention, selecting the isolated T cells which have the gene expression profile comprises carrying out single cell transcriptome analysis.

Detecting the expression and/or non-expression of one or more genes by the one or more single T cells may be carried out using, for example, the CHROMIUM Single Cell Gene Expression Solution system (10x Genomics, Pleasanton, CA) (“CHROMIUM system”). The CHROMIUM system performs deep profiling of complex cell populations with high-throughput digital gene expression on a cell-by-cell basis. The CHROMIUM system barcodes the cDNA of individual cells for 5′ transcriptional or TCR analysis. For example, samples may start with an input of 10,000 cells and yield data for about 3000 cells/sample, with an average of about 500 genes/cell.

In an aspect of the invention, selecting the isolated T cells which have the gene expression profile comprises carrying out Cellular Indexing of Transcriptomes and Epitopes by Sequencing (CITE-Seq) analysis. CITE-Seq is described at, for example, Stoeckius et al., Nat. Methods, 14(9): 865-868 (2017). Briefly, CITE-seq combines antibody-based detection of protein markers together with transcriptome profiling for many single cells in parallel. Oligonucleotide-labeled antibodies are used to integrate cellular protein and transcriptome measurements into an efficient, single-cell readout.

Because of the high dimensionality of the data yielded by the single cell transcriptome analysis (e.g., about 3000 cells/sample and about 500 genes/cell), dimensionality reduction may be carried out for analysis of the gene expression data. Accordingly, in an aspect of the invention, selecting the isolated T cells which have the gene expression profile comprises carrying out one or more single cell dimensional reduction methods. An example of a single cell dimensional reduction method is t-Distributed Stochastic Neighbor Embedding (t-SNE) analysis. t-SNE visualizes high-dimensional data by giving each data point a location in a two or three-dimensional map. t-SNE is described at, for example, Van der Maaten and Hinton, J. Machine Learning Res., 9: 2579-2605 (2008). Briefly, t-SNE is carried out in two steps. In step 1, a probability distribution is created in the high-dimensional space that dictates the relationships between various neighboring points. In step 2, a low dimensional space is recreated that follows that probability distribution as best as possible. The “t” in t-SNE comes from the t-distribution, which is the distribution used in Step 2. The “S” and “N” (“stochastic” and “neighbor”) come from the use of a probability distribution across neighboring points. Another example of a single cell dimensional reduction method is Uniform Manifold Approximation and Projection (UMAP).

The gene expression profile may include (i) positive expression of one or more genes, (ii) negative expression of one or more genes, or (iii) positive expression of one or more genes in combination with negative expression of one or more genes. As used herein, the term “positive” (which may be abbreviated as “⁺”), with reference to expression of the indicated gene, means that the T cell upregulates expression of the indicated gene as compared to other T cells in the tumor sample of the patient. Upregulated expression may encompass, for example, a quantitative increase in expression of the indicated gene by an average logarithmic fold change (to the base 2) of about 0.2, about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, or a range of any two of the foregoing values, or more. The term “negative” (which may be abbreviated as “-”), as used herein with reference to expression of the indicated gene, means that the T cell downregulates expression of the indicated gene as compared to other T cells in the tumor sample of the patient. Downregulated expression may encompass, for example, a quantitative decrease in expression of the indicated gene by an average logarithmic fold change (to the base 2) of about −0.2, about −0.5, about −1, about −2, about −3, about −4, about −5, about −6, about −7, about −8, about −9, about −10, about −11, about −12, about −13, about −14, about −15, about −16, about −17, about −18, about −19, about −20, about −21, about −22, about −23, about −24, about −25, about −26, about −27, about −28, about −29, about −30, about −31, about −32, about −33, about −34, about −35, or a range of any two of the foregoing values, or more. Although downregulated expression may encompass an absence of expression of the indicated gene, downregulation also encompasses the presence of the expression of the indicated gene, albeit at a lower level as compared to other T cells in the tumor sample of the patient.

In an aspect of the invention, the gene expression profile comprises: (i) one or both of CD4⁺ and CD8⁺ and (ii) one or more of AFAP1IL2⁺, ASB2⁺, CXCL13⁺, HMOX1⁺, ITM2A⁺, KLRB1⁺, PDLIM4⁺, TIGIT⁺, LTB⁻, LYAR⁻, RGCC⁻, and S100A10⁻. For example, the gene expression profile may comprise: (i) one or both of CD4⁺ and CD8⁺ and (ii) any 1, 2, 3, 4, 5, 6, 7, 8, 9, 10, 11, or more (or a range between any two of the foregoing values) of AFAP1IL2⁺, ASB2⁺, CXCL13⁺, HMOX1⁺, ITM2A⁺, KLRB1⁺, PDLIM4⁺, LTB⁻, LYAR⁻, RGCC⁻, and S100A10⁻. In an aspect of the invention, the gene expression profile comprises (i) one or both of CD4⁺ and CD8+ and (ii) all of AFAP1IL2⁺, ASB2⁺, CXCL13⁺, HMOX1⁺, ITM2A⁺, KLRB1⁺, PDLIM4⁺, LTB⁻, LYAR⁻, RGCC⁻, and S100A10⁻.

In another aspect of the invention, the gene expression profile comprises: CD4⁺ and one or more of BATF⁺, CD247⁺, CXCL13⁺, DNPH1⁺, DUSP4⁺, GYPC⁺, IFITM1⁺, IGFLR1⁺, ITM2A⁺, KLRB1⁺, LIMS1⁺, NMB⁺, NR3C1⁺, SH2D1A⁺, SPOCK2⁺, SUPT3H⁺, TIGIT⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻. The gene expression profile may comprise, for example, (i) CD4⁺ and CXCL13⁺; (ii) CD4⁺, CXCL13⁺, and one or more of CD39⁺, TIGIT⁺, and PD-1⁻; or (iii) CD4⁺, CXCL13⁺, CD39⁺, TIGIT⁺, and PD-1⁻. The gene expression profile may comprise: CD4⁺ and any 1, 2, 3, 4, 5, 6, 7, 8, 9, 10, 11, 12, 13, 14, 15, 16, 17, 18, 19, 20, 21, 22, 23, 24, 25, 26, 27, 28, 29, or more (or a range between any two of the foregoing values) of BATF⁺, CD247⁺, CXCL13⁺, DNPH1⁺, DUSP4⁺, GYPC⁺, IFITM1⁺, IGFLR1⁺, ITM2A⁺, KLRB1⁺, LIMS1⁺, NMB⁺, NR3C1⁺, SH2D1A⁺, SPOCK2⁺, SUPT3H⁺, TIGIT⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻. In an aspect of the invention, the gene expression profile comprises CD4⁺ and all of BATF⁺, CD247⁺, CXCL13⁺, DNPH1⁺, DUSP4⁺, GYPC⁺, IFITM1⁺, IGFLR1⁺, ITM2A⁺, KLRB1⁺, LIMS1⁺, NMB⁺, NR3C1⁺, SH2D1A⁺, SPOCK2⁺, SUPT3H⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻.

In still another aspect of the invention, the gene expression profile comprises: CD8⁺ and one or more of AFAP1IL2⁺, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA1⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, ANXA1⁻, EEF1B2⁻, EMP3⁻, IL7R⁻, LGALS3⁻, LTB⁻, LYAR⁻, RGCC⁻, RPL36A⁻, and S100A10⁻. The gene expression profile may comprise, for example, (i) CD8⁺ and CXCL13⁺; (ii) CD8⁺, TIGIT⁺, and one or both of CD39⁺ and PD-1⁺; (iii) CD8⁺, TIGIT⁺, CD39⁺, and PD-1⁺; (iv) CD8⁺, CXCL13⁺, and one or more of CD39⁺, TIGIT⁺, and PD-1⁺; or (v) CD8⁺, CXCL13⁺, CD39⁺, TIGIT⁺, and PD-1⁺. For example, the gene expression profile may comprise: CD8⁺ and any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or more (or a range between any two of the foregoing values) of AFAP1IL2⁺, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA1⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, TIGIT⁺, ANXA1⁻, EEF1B2⁻, EMP3⁻, IL7R⁻, LGALS3⁻, LTB⁻, LYAR⁻, RGCC⁻, RPL36A⁻, and S100A10⁻. In an aspect of the invention, the gene expression profile comprises CD8⁺ and all of AFAP1IL2+, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA1⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, TIGIT⁺, ANXA1⁻, EEF1B2⁻, EMP3⁻, IL7R⁻, LGALS3⁻, LTB⁻, LYAR⁻, RGCC⁻, RPL36A⁻, and S100A10⁻.

In an aspect of the invention, the gene expression profile comprises one or more of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LAG3⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺, SMC4⁺, SUPT3H⁺, TNFRSF18⁺, TOX⁺, TRAF3IP3⁺, and YPEL2⁺. For example, the gene expression profile may comprise: any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or more of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LAG3⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺, SMC4⁺, SUPT3H⁺, TIGIT⁺, TNFRSF18⁺, TOX⁺, TRAF3IP3⁺, and YPEL2⁺. In an aspect of the invention, the gene expression profile comprises all of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LAG3⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺, SMC4⁺, SUPT3H⁺, TNFRSF18⁺, TOX⁺, TRAF3IP3⁺, and YPEL2⁺. In an aspect of the invention, the gene expression profile further comprises LAG3⁺.

In an aspect of the invention, the gene expression profile comprises CD4⁺ and one or more of ADI1⁺, AHI1⁺, ARID5B⁺, BATF⁺, CMTM7⁺, CPM⁺, CXCL13⁺, CYTH1⁺, ELMO1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TIGIT⁺, TLK1⁺, TMEM123⁺, TMEM70⁺, TNIK⁺, TOX⁺, TSHZ2⁺, UCP2⁺, VOPP1⁺, and YPEL2⁺. For example, the gene expression profile may comprise: any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more of ADI1⁺, AHI1⁺, ARID5B⁺, BATF⁺, CMTM7⁺, CPM⁺, CXCL13⁺, CYTH1⁺, ELMO1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TIGIT⁺, TLK1⁺, TMEM123⁺, TMEM70⁺, TNIK⁺, TOX⁺, TSHZ2⁺, UCP2⁺, VOPP1⁺, and YPEL2⁺. In an aspect of the invention, the gene expression profile comprises CD4⁺ and all of ADI1⁺, AHI1⁺, ARID5B⁺, BATF⁺, CMTM7⁺, CPM⁺, CXCL13⁺, CYTH1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TIGIT⁺, TLK1⁺, TMEM123⁺, TMEM70⁺, TNIK⁺, TOX⁺, TSHZ2⁺, UCP2⁺, VOPP1⁺, and YPEL2⁺.

In an aspect of the invention, the gene expression profile comprises CD8⁺ and one or more of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺. For example, the gene expression profile may comprise: any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺. In an aspect of the invention, the gene expression profile comprises CD8⁺ and all of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺. In an aspect of the invention, the gene expression profile further comprises LAG3⁺.

In an aspect of the invention, the gene expression profile comprises one or more of AHI1⁺, CXCL13⁺, FABP5⁺, NAP1L4⁺, ORMDL3⁺, PPP1R16B⁺, SH2D1A⁺, TIGIT⁺, and TOX⁺. For example, the gene expression profile may comprise: any 1, 2, 3, 4, 5, 6, 7, 8, or more of AHI1⁺, CXCL13⁺, FABP5⁺, NAP1L4⁺, ORMDL3⁺, PPP1R16B⁺, SH2D1A⁺, TIGIT⁺, and TOX⁺. In an aspect of the invention, the gene expression profile comprises all of AHI1⁺, CXCL13⁺, FABP5⁺, NAP1L4⁺, ORMDL3⁺, PPP1R16B⁺, SH2D1A⁺, TIGIT⁺, and TOX⁺.

In an aspect of the invention, the gene expression profile comprises one or more of TIGIT⁺, CD39⁺, and PD-1⁺. For example, the gene expression profile may comprise: any 1, 2, or more of TIGIT⁺, CD39⁺, and PD-1⁺. In an aspect of the invention, the gene expression profile comprises all of TIGIT⁺, CD39⁺, and PD-1⁺.

In still another aspect of the invention, the gene expression profile comprises: CD8⁺ and one or more of CD39⁺, CD74⁺, CD103⁺, CD106⁺, CD137⁺, HLA-DR⁺, TGIT⁺, CCR7⁻, CD8A⁻, CD16⁻, CD45RA⁻, CD62L⁻ and IL7R⁻. In an aspect of the invention, the gene expression profile further comprises one or both of PD-1⁺ and TIM-3⁺. For example, the gene expression profile may comprise: CD8⁺ and any 1, 2, 3, 4, 5, 6, 7, 8, 9, 10, 11, 12, or more (or a range between any two of the foregoing values) of CD39⁺, CD74⁺, CD103⁺, CD106⁺, CD137⁺, HLA-DR⁺, TIGIT⁺, CCR7⁻, CD8A⁻, CD16⁻, CD45RA⁻, CD62L⁻ and IL7R⁻. In an aspect of the invention, the gene expression profile comprises: CD8⁺ and all of CD39⁺, CD74⁺, CD103⁺, CD106⁺, CD137⁺, HLA-DR⁺, CCR7⁻, CD8A⁻, CD16⁻, CD45RA⁻, CD62L⁻ and IL7R⁻. In an aspect of the invention, the gene expression profile comprises one or more of (as compared with other CD8⁺ T-cells in the tumor): CD8A low, CD45RA negative, CD62L negative to very low, CCR7 negative to very low, CD16 negative to very low, and IL7R negative to very low. In an aspect of the invention, the gene expression profile comprises: CD8⁺ and one or more of cell surface proteins CD39⁺, CD74⁺, CD103⁺, CD106⁺, CD137⁺, HLA-DR⁺, CCR7^{lo}, CD8A^{lo}, CD16^{lo}, CD45RA^{lo}, CD62L^{lo} and IL7R^{lo}. The term “low” (which may be abbreviated as “lo”), as used herein with reference to expression of the indicated gene, refers to a subset of cells that stain less brightly for the indicated expressed gene using immunohistochemical methods (e.g., FACS, flow cytometry, immunofluorescence assays and microscopy) than other cells that are positive for expression of the indicated gene. For example, cells with a “low” level of expression of the indicated gene may stain less brightly than about 50%, about 60%, about 70%, about 80%, about 90%, or about 95%, or a range of any two of the foregoing values, of the other cells that are positive for expression of the indicated gene.

In an aspect of the invention, the gene expression profile comprises TIGIT⁺. In another aspect of the invention, the gene expression profile comprises CXCL13⁺.

Selecting the isolated T cells which have the gene expression profile may comprise detecting the presence or absence of, or measuring the quantity of, the product(s) of expression of the gene(s) in the gene expression profiles described herein. In this regard, selecting the isolated T cells which have the gene expression profile may comprise detecting the presence of protein(s) encoded by positively expressed gene(s) of the gene expression profile. Alternatively or additionally, selecting the isolated T cells which have the gene expression profile may comprise detecting the absence of protein(s) encoded by gene(s) that are negative for expression in the gene expression profile. Alternatively or additionally, selecting the isolated T cells which have the gene expression profile may comprise measuring the quantity of protein(s) encoded by gene(s) that are negative for expression in the gene expression profile. Alternatively or additionally, selecting the isolated T cells which have the gene expression profile may comprise measuring the quantity of protein(s) encoded by gene(s) that are positive for expression in the gene expression profile. Alternatively or additionally, selecting the isolated T cells which have the gene expression profile may comprise detecting the presence of RNA encoded by positively expressed gene(s) of the gene expression profile. Alternatively or additionally, selecting the isolated T cells which have the gene expression profile may comprise detecting the absence of RNA encoded by gene(s) that are negative for expression in the gene expression profile. Alternatively or additionally, selecting the isolated T cells which have the gene expression profile may comprise measuring the quantity of RNA encoded by positively expressed gene(s) of the gene expression profile. Alternatively or additionally, selecting the isolated T cells which have the gene expression profile may comprise measuring the quantity of RNA encoded by negatively expressed gene(s) of the gene expression profile. In an aspect of the invention, selecting the isolated T cells which have the gene expression profile comprises detecting the presence and/or absence of cell surface expression of the one or more genes in the gene expression profile. In an aspect of the invention, selecting the isolated T cells which have the gene expression profile comprises measuring the quantity of cell surface expression of the one or more genes in the gene expression profile. Cell surface expression may be detected or measured by any suitable method, for example, flow cytometry (e.g., fluorescence-activated cell sorting (FACS)).

In an aspect of the invention, the method of preparing an enriched population of T cells having antigenic specificity for a target antigen does not comprise expanding the numbers of the T cells. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). For example, expansion of the numbers of T cells is carried out by culturing the T cells with OKT3 antibody, IL-2, and feeder PBMC (e.g., irradiated allogeneic PBMC). Rare and/or fragile T cells with the desired specificity for a target antigen may be lost during expansion of the numbers of T cells. The inventive methods may, advantageously, prepare an enriched population of T cells having antigenic specificity for a target antigen including such rare and/or fragile T cells by carrying out the inventive methods without expanding the numbers of the T cells.

The method may further comprise separating the selected T cells from the unselected cells, wherein the separated selected T cells provide an enriched population of T cells having antigenic specificity for the target antigen. In this regard, the selected cells may be physically separated from unselected cells, i.e., the cells that do not have the gene expression profile. The selected cells may be separated from unselected cells by any suitable method such as, for example, sorting.

Another aspect of the invention provides a method of isolating a T cell receptor (TCR), or an antigen-binding portion thereof, having antigenic specificity for the target antigen.

The “antigen-binding portion” of the TCR, as used herein, refers to any portion comprising contiguous amino acids of the TCR of which it is a part, provided that the antigen-binding portion specifically binds to the target antigen as described herein with respect to other aspects of the invention. The term “antigen-binding portion” refers to any part or fragment of the TCR of the invention, which part or fragment retains the biological activity of the TCR of which it is a part (the parent TCR). Antigen-binding portions encompass, for example, those parts of a TCR that retain the ability to specifically bind to the target antigen, or detect, treat, or prevent a condition, to a similar extent, the same extent, or to a higher extent, as compared to the parent TCR. In reference to the parent TCR, the functional portion can comprise, for instance, about 10%, 25%, 30%, 50%, 68%, 80%, 90%, 95%, or more, of the parent TCR.

The antigen-binding portion can comprise an antigen-binding portion of either or both of the α and β chains of the TCR of the invention, such as a portion comprising one or more of the complementarity determining region (CDR)1, CDR2, and CDR3 of the variable region(s) of the α chain and/or β chain of the TCR of the invention. In an aspect of the invention, the antigen-binding portion can comprise the amino acid sequence of the CDR1 of the α chain (CDR1α), the CDR2 of the a chain (CDR2α), the CDR3 of the α chain (CDR3α), the CDR1 of the β chain (CDR1β), the CDR2 of the β chain (CDR2β), the CDR3 of the β chain (CDR3β), or any combination thereof. Preferably, the antigen-binding portion comprises the amino acid sequences of CDR1α, CDR2α, and CDR3α; the amino acid sequences of CDR1β, CDR2β, and CDR3β; or the amino acid sequences of all of CDR1α, CDR2α, CDR3α, CDR1β, CDR2β, and CDR3β of the inventive TCR.

In an aspect of the invention, the antigen-binding portion can comprise, for instance, the variable region of the inventive TCR comprising a combination of the CDR regions set forth above. In this regard, the antigen-binding portion can comprise the amino acid sequence of the variable region of the α chain (Vα), the amino acid sequence of the variable region of the β chain (Vβ), or the amino acid sequences of both of the Vα and Vβ of the inventive TCR.

In an aspect of the invention, the antigen-binding portion may comprise a combination of a variable region and a constant region. In this regard, the antigen-binding portion can comprise the entire length of the α or β chain, or both of the α and β chains, of the inventive TCR.

The method may comprise preparing an enriched population of T cells having antigenic specificity for the target antigen according to any of the inventive methods described herein with respect to other aspects of the invention.

The method may comprise sorting the T cells in the enriched population into separate single T cell samples and sequencing TCR alpha chain CDR3 and beta chain CDR3 in one or more of the separate single T cell samples. In an aspect of the invention, the sequencing of the TCR alpha chain CDR3 and beta chain CDR3 may be carried out using the single cell transcriptome analysis employed for the analyzing the gene expression profile described herein with respect to other aspects of the invention. Other techniques for sequencing the TCR alpha chain CDR3 and beta chain CDR3 are described at, for example, US 2020/0056237 and WO 2017/048614.

The method may further comprise pairing an alpha chain variable region comprising a CDR3 with a beta chain variable region comprising a CDR3 encoded by the nucleic acid of the separate single T cell samples. In this regard, the method may comprise reconstructing the TCR so that the pairing of the alpha chain variable region comprising a CDR3 with the beta chain variable region comprising a CDR3 yields a functional TCR. In an aspect of the invention, the TCR is reconstructed in silico. Methods of reconstructing the TCR in silico and pairing an alpha chain variable region comprising a CDR3 with a beta chain variable region comprising a CDR3 are described at, for example, US 2020/0056237 and WO 2017/048614.

The method may comprise isolating a nucleotide sequence that encodes the TCR, or the antigen-binding portion thereof, from the selected T cells, wherein the TCR, or the antigen-binding portion thereof, has antigenic specificity for the target antigen.

The method may comprise introducing a nucleotide sequence encoding the paired alpha chain variable region and beta chain variable region into host cells and expressing the paired alpha chain variable region and beta chain variable region by the host cells. Introducing the nucleotide sequence (e.g., a recombinant expression vector) encoding the isolated TCR, or the antigen-binding portion thereof, into host cells may be carried out in any of a variety of different ways known in the art as described in, e.g., Green et al. (Eds.), Molecular Cloning: A Laboratory Manual, Cold Spring Harbor Laboratory Press; 4th Ed. (2012). Non-limiting examples of techniques that are useful for introducing a nucleotide sequence into host cells include transformation, transduction, transfection, and electroporation.

In an aspect of the invention, the method may comprise cloning the nucleotide sequence that encodes the TCR, or the antigen-binding portion thereof, into a recombinant expression vector using established molecular cloning techniques as described in, e.g., Green et al., supra. The recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of transposon/transposase, the pUC series (Fermentas Life Sciences), the pBluescript series (Stratagene, LaJolla, CA), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In other aspects, the recombinant expression vector is a lentiviral vector or a transposon.

The host cell(s) can be a eukaryotic cell, e.g., plant, animal, fungi, or algae, or can be a prokaryotic cell, e.g., bacteria or protozoa. The host cell(s) can be a cultured cell or a primary cell, i.e., isolated directly from an organism, e.g., a human. The host cell(s) can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating a nucleotide sequence encoding the TCR, or antigen-binding portion thereof, the host cell is preferably a prokaryotic cell, e.g., a DH5α cell. For purposes of producing a recombinant TCR, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Thi and Thz cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

The method may comprise screening the host cells expressing the paired alpha chain variable region and beta chain variable region for antigenic specificity for the target antigen and selecting the paired alpha chain variable region and beta chain variable region that have antigenic specificity for the target antigen, wherein the TCR, or an antigen-binding portion thereof, having antigenic specificity for the target antigen is isolated. The screening of the host cells for antigenic specificity and selecting the paired alpha chain variable region and beta chain variable region that have antigenic specificity may be carried out using known techniques as described, for example, in US 2017/0218042 and US 2017/0224800.Further aspects of the invention may provide a method of obtaining target antigen-specific TCRs by, for example, single cell RNA sequencing and subsequent application of the gene expression profiles to in silico reconstruct the TCRs. Accordingly, an aspect of the invention provides a method of isolating a TCR, or an antigen-binding portion thereof, having antigenic specificity for a target antigen, the method comprising: isolating T cells from a tumor sample of a patient; sorting the T cells in the enriched population into separate single T cell samples; sequencing TCR CDR3 in the separate single T cell samples; selecting the separate single T cell samples which have a gene expression profile; pairing an alpha chain variable region comprising a CDR3 with a beta chain variable region comprising a CDR3 encoded by the nucleic acid of the separate single T cell samples with the gene expression profile; introducing a nucleotide sequence encoding the paired alpha chain variable region and beta chain variable region into host cells and expressing the paired alpha chain variable region and beta chain variable region by the host cells; screening the host cells expressing the paired alpha chain variable region and beta chain variable region for antigenic specificity for the target antigen; and selecting the paired alpha chain variable region and beta chain variable region that have antigenic specificity for the target antigen, wherein the TCR, or an antigen-binding portion thereof, having antigenic specificity for the target antigen is isolated. The isolating of the T cells, sorting of the T cells, sequencing of the TCR CDR3, selecting of the separate single T cell samples, pairing of the alpha and beta chain variable region, introducing of the nucleotide sequence into host cells, screening of the host cells, the selecting of the paired alpha and beta chain variable regions, and the gene expression profile may be any of the gene expression profiles described herein with respect to other aspects of the invention.

The TCR, or the antigen-binding portion thereof, isolated by the inventive methods may be useful for preparing cells for adoptive cell therapies. In this regard, an aspect of the invention provides a method of preparing a population of cells that express a TCR, or an antigen-binding portion thereof, having antigenic specificity for a target antigen, the method comprising isolating a TCR, or an antigen-binding portion thereof, as described herein with respect to other aspects of the invention, and introducing the nucleotide sequence encoding the isolated TCR, or the antigen-binding portion thereof, into PBMC to obtain cells that express the TCR, or the antigen-binding portion thereof

Introducing the nucleotide sequence (e.g., a recombinant expression vector) encoding the isolated TCR, or the antigen-binding portion thereof, into PBMC may be carried out in any of a variety of different ways known in the art as described in, e.g., Green et al. supra. Non-limiting examples of techniques that are useful for introducing a nucleotide sequence into PBMC include transformation, transduction, transfection, and electroporation.

In an aspect of the invention, the method comprises introducing the nucleotide sequence encoding the isolated TCR, or the antigen-binding portion thereof, into PBMC that are autologous to the patient. In this regard, the TCRs, or the antigen-binding portions thereof, identified and isolated by the inventive methods may be personalized to each patient. However, in another aspect, the inventive methods may identify and isolate TCRs, or the antigen-binding portions thereof, that have antigenic specificity against a mutated amino acid sequence that is encoded by a recurrent (also referred to as “hot-spot”) cancer-specific mutation. In this regard, the method may comprise introducing the nucleotide sequence encoding the isolated TCR, or the antigen-binding portion thereof, into PBMC that are allogeneic to the patient. For example, the method may comprise introducing the nucleotide sequence encoding the isolated TCR, or the antigen-binding portion thereof, into the PBMC of another patient whose tumors express the same mutation in the context of the same MHC molecule.

In an aspect of the invention, the PBMC include T cells. The T cells may be any type of T cell, for example, any of those described herein with respect to other aspects of the invention. Without being bound to a particular theory or mechanism, it is believed that less differentiated, “younger” T cells may be associated with any one or more of greater in vivo persistence, proliferation, and antitumor activity as compared to more differentiated, “older” T cells. Accordingly, the inventive methods may, advantageously, identify and isolate a TCR, or an antigen-binding portion thereof, that has antigenic specificity for the target antigen and introduce the TCR, or an antigen-binding portion thereof, into “younger” T cells that may provide any one or more of greater in vivo persistence, proliferation, and antitumor activity as compared to “older” T cells (e.g., effector cells in a patient's tumor) from which the TCR, or the antigen-binding portion thereof, may have been isolated.

The inventive methods may, advantageously collect more than one or all of the TCRs that are identified as having a gene expression profile described herein, e.g., by single cell transcriptomics, pool all these TCRs and combine them as a clinical T cell therapy product. In this regard, another aspect of the invention provides a method of preparing a pooled population of cells that express a TCR, or an antigen-binding portion thereof, having antigenic specificity for a target antigen. The method may comprise (a) preparing an enriched population of T cells having antigenic specificity for the target antigen according to any of the inventive methods described herein; (b) sorting the T cells in the enriched population into separate single T cell samples; (c) sequencing TCR complementarity determining regions 3 (CDR3) in the separate single T cell samples; (d) pairing an alpha chain variable region comprising a CDR3 with a beta chain variable region comprising a CDR3 encoded by the nucleic acid of the separate single T cell samples; (e) introducing a nucleotide sequence encoding the paired alpha chain variable region and beta chain variable region into peripheral blood mononuclear cells (PBMC) and expressing the paired alpha chain variable region and beta chain variable region by the PBMC; and carrying out the sequencing, pairing, and introducing of the nucleotide sequence for a plurality of the separate single T cell samples of the enriched population of T cells having antigenic specificity for the target antigen prepared according to any of the inventive methods described herein, thereby providing a pooled population of cells that express a TCR, or an antigen-binding portion thereof, having antigenic specificity for a target antigen. The sorting, sequencing, pairing and introducing of the nucleotide sequence may be carried out as described herein with respect to other aspects of the invention.

In an aspect of the invention, the method of preparing a population of cells that express a TCR, or an antigen-binding portion thereof, further comprises expanding the numbers of PBMC that express the TCR, or the antigen-binding portion thereof. Expanding the numbers of PBMC may be carried out as described herein with respect to other aspects of the invention. In an aspect of the invention, the method of preparing a population of cells that express a TCR, or an antigen-binding portion thereof, comprises expanding the numbers of PBMC that express the TCR, or the antigen-binding portion thereof, while the method of preparing an enriched population of T cells having antigenic specificity for a target antigen does not comprise expanding the numbers of T cells.

Another aspect of the invention provides a TCR, or an antigen-binding portion thereof, isolated by any of the methods described herein with respect to other aspects of the invention. An aspect of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof Another aspect of the invention provides an antigen-binding portion of the TCR comprising one or more CDR regions, one or more variable regions, or one or both of the α and β chains of the TCR, as described herein with respect to other aspects of the invention. The polypeptides of the inventive TCR, or the antigen-binding portion thereof, can comprise any amino acid sequence, provided that the TCR, or the antigen-binding portion thereof, has antigenic specificity for the target antigen.

Another aspect of the invention provides an isolated population of cells prepared according to any of the methods described herein with respect to other aspects of the invention. The population of cells can be a heterogeneous population comprising the PBMC expressing the isolated TCR, or the antigen-binding portion thereof, in addition to at least one other cell, e.g., a host cell (e.g., a PBMC), which does not express the isolated TCR, or the antigen-binding portion thereof, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of PBMC (e.g., consisting essentially of) expressing the isolated TCR, or the antigen-binding portion thereof. The population also can be a clonal population of cells, in which all cells of the population are clones of a single PBMC expressing the isolated TCR, or the antigen-binding portion thereof, such that all cells of the population express the isolated TCR, or the antigen-binding portion thereof. In one aspect of the invention, the population of cells is a clonal population comprising PBMC expressing the isolated TCR, or the antigen-binding portion thereof, as described herein. By introducing the nucleotide sequence encoding the isolated TCR, or the antigen binding portion thereof, into PBMC, the inventive methods may, advantageously, provide a population of cells that comprises a high proportion of PBMC cells that express the isolated TCR and have antigenic specificity for the target antigen. In an aspect of the invention, about 1% to about 100%, for example, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, or a range defined by any two of the foregoing values, of the population of cells comprises PBMC cells that express the isolated TCR and have antigenic specificity for the target antigen. Without being bound to a particular theory or mechanism, it is believed that populations of cells that comprise a high proportion of PBMC cells that express the isolated TCR and have antigenic specificity for the target antigen have a lower proportion of irrelevant cells that may hinder the function of the PBMC, e.g., the ability of the PBMC to target the destruction of target cells and/or treat or prevent a condition. Target cells may include, for example, cancer cells or virus-infected cells.

The inventive TCRs, or the antigen-binding portions thereof, and populations of cells can be formulated into a composition, such as a pharmaceutical composition. In this regard, the invention provides a pharmaceutical composition comprising any of the inventive TCRs, or the antigen-binding portions thereof, or populations of cells and a pharmaceutically acceptable carrier. The inventive pharmaceutical composition can comprise an inventive TCR, or an antigen-binding portion thereof, or population of cells in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR, or the antigen-binding portion thereof, or population of cells under consideration. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular inventive TCR, the antigen-binding portion thereof, or population of cells, as well as by the particular method used to administer the inventive TCR, the antigen-binding portion thereof, or population of cells. Accordingly, there are a variety of suitable formulations of the pharmaceutical composition of the invention. Suitable formulations may include any of those for intratumoral, oral, parenteral, subcutaneous, intravenous, intramuscular, intraarterial, intrathecal, or interperitoneal administration. More than one route can be used to administer the inventive TCR or population of cells, and in certain instances, a particular route can provide a more immediate and more effective response than another route.

Preferably, the inventive TCR, the antigen-binding portion thereof, or population of cells is administered by injection, e.g., intravenously. When the inventive population of cells is to be administered, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, IL), about 5% dextrose in water, or Ringer's lactate. In an aspect, the pharmaceutically acceptable carrier is supplemented with human serum albumin.

It is contemplated that the inventive TCRs, the antigen-binding portions thereof, populations of cells, and pharmaceutical compositions can be used in methods of treating or preventing a condition. Without being bound to a particular theory or mechanism, the inventive TCRs, or the antigen-binding portions thereof, are believed to bind specifically to a target antigen, such that the TCR, or the antigen-binding portion thereof, when expressed by a cell, is able to mediate an immune response against a target cell expressing the target antigen. In this regard, the invention provides a method of treating or preventing a condition in a mammal comprising (i) preparing an enriched population of T cells having antigenic specificity for a target antigen according to any of the methods described herein with respect to other aspects of the invention; and administering the population of cells to the mammal in an amount effective to treat or prevent the condition in the mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a condition in a mammal. Furthermore, the treatment or prevention provided by the inventive method can include treatment or prevention of one or more signs or symptoms of the condition being treated or prevented. For example, treatment or prevention can include promoting the regression of a tumor. Also, for purposes herein, “prevention” can encompass delaying the onset of the condition, or a symptom, sign, or recurrence thereof

For purposes of the invention, the amount or dose of the inventive TCR, the antigen-binding portion thereof, population of cells, or pharmaceutical composition administered (e.g., numbers of cells when the inventive population of cells is administered) should be sufficient to effect, e.g., a therapeutic or prophylactic response, in the mammal over a reasonable time frame. For example, the dose of the inventive TCR, the antigen-binding portion thereof, population of cells, or pharmaceutical composition should be sufficient to bind to the target antigen, or detect, treat or prevent the condition in a period of from about 2 hours or longer, e.g., 12 to 24 or more hours, from the time of administration. In certain aspects, the time period could be even longer. The dose will be determined by the efficacy of the particular inventive TCR, the antigen-binding portion thereof, population of cells, or pharmaceutical composition administered and the condition of the mammal (e.g., human), as well as the body weight of the mammal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, or the antigen-binding portion thereof, upon administration of a given dose of such T cells to a mammal among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR, the antigen-binding portion thereof, population of cells, or pharmaceutical composition also will be determined by the existence, nature and extent of any adverse side effects that might accompany the administration of a particular inventive TCR, the antigen-binding portion thereof, population of cells, or pharmaceutical composition. Typically, the attending physician will decide the dosage of the inventive TCR, the antigen-binding portion thereof, population of cells, or pharmaceutical composition with which to treat each individual patient, taking into consideration a variety of factors, such as age, body weight, general health, diet, sex, inventive TCR, the antigen-binding portion thereof, population of cells, or pharmaceutical composition to be administered, route of administration, and the severity of the condition being treated.

In an aspect in which the inventive population of cells is to be administered, the number of cells administered per infusion may vary, for example, in the range of one million to 100 billion cells; however, amounts below or above this exemplary range are within the scope of the invention. For example, the daily dose of inventive host cells can be about 1 million to about 150 billion cells (e.g., about 5 million cells, about 25 million cells, about 500 million cells, about 1 billion cells, about 5 billion cells, about 20 billion cells, about 30 billion cells, about 40 billion cells, about 60 billion cells, about 80 billion cells, about 100 billion cells, about 120 billion cells, about 130 billion cells, about 150 billion cells, or a range defined by any two of the foregoing values), preferably about 10 million to about 130 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, about 100 billion cells, about 110 billion cells, about 120 billion cells, about 130 billion cells, or a range defined by any two of the foregoing values), more preferably about 100 million cells to about 130 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, about 50 billion cells, about 75 billion cells, about 90 billion cells, about 100 billion cells, about 110 billion cells, about 120 billion cells, about 130 billion cells, or a range defined by any two of the foregoing values).

For purposes of the inventive methods, wherein populations of cells are administered, the cells can be cells that are allogeneic or autologous to the mammal. Preferably, the cells are autologous to the mammal.

Another aspect of the invention provides a method of preparing a medicament for the treatment or prevention of a condition in a mammal, the method comprising (i) preparing an enriched population of T cells having antigenic specificity for a target antigen according to any of the methods described herein with respect to other aspects of the invention; or (ii) preparing an isolated population of cells that express a TCR, or an antigen-binding portion thereof, according to any of the methods described herein with respect to other aspects of the invention.

In an aspect of the invention, the condition is cancer. The cancer may, advantageously, be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, cholangiocarcinoma, chronic lymphocytic leukemia, chronic myeloid cancer, colon cancer, esophageal cancer, uterine cervical cancer, gastrointestinal carcinoid tumor, glioma, Hodgkin lymphoma, hypopharynx cancer, kidney cancer, larynx cancer, liver cancer, lung cancer (e.g., non-small cell lung cancer), malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, urinary bladder cancer, solid tumors, and liquid tumors. Preferably, the cancer is an epithelial cancer. In an aspect, the cancer is cholangiocarcinoma, melanoma, colon cancer, lung cancer, breast cancer, or rectal cancer.

In an aspect of the invention, the condition is a viral condition. For purposes herein, “viral condition” means a condition that can be transmitted from person to person or from organism to organism, and is caused by a virus. In an aspect of the invention, the viral condition is caused by a virus selected from the group consisting of herpes viruses, pox viruses, hepadnaviruses, papilloma viruses, adenoviruses, coronoviruses, orthomyxoviruses, paramyxoviruses, flaviviruses, and caliciviruses. For example, the viral condition may be caused by a virus selected from the group consisting of respiratory syncytial virus (RSV), influenza virus, herpes simplex virus, Epstein-Barr virus, HPV, varicella virus, cytomegalovirus, hepatitis A virus, hepatitis B virus, hepatitis C virus, human immunodeficiency virus (HIV), human T-lymphotropic virus, calicivirus, adenovirus, and Arena virus. In an aspect of the invention, the viral condition may be a chronic viral infection caused by any of the viruses described herein. The viral condition may be, for example, influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, HIV/AIDS, HPV infection, and mononucleosis. In an embodiment of the invention, the viral condition is a viral infection caused by a cancer-associated virus.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). Preferably, the mammals are from the order Artiodactyla, including Bovines (cows) and Swines (pigs) or of the order Perssodactyla, including Equines (horses). Preferably, the mammals are of the order Primates, Ceboids, or Simoids (monkeys) or of the order Anthropoids (humans and apes). A more preferred mammal is the human. In an especially preferred aspect, the mammal is the patient expressing the target antigen.

The following examples further illustrate the invention but, of course, should not be construed as in any way limiting its scope.

### EXAMPLES

The following materials and methods were employed in the experiments described in Examples 1-12.

### Experimental Setup/Sample Preparation

Samples for 10× Genomics 5′ Single Cell Gene Expression Profiling and TCR sequencing (10×scTCR)/transcriptome analysis were prepared consistently in the following manner. Single cell suspensions were made from TIL harvest and cryopreserved. Samples were thawed and rested overnight in TIL media without cytokines. CD4 positive and/or CD8 positive, viable cells were isolated using a Sony cell sorter (MA900 or SH800), usually ˜30,000 total T cells. Samples were delivered to the Single Cell Analysis Core Facility, NIH (SCAF) for the 10×scTCR analysis. SCAF delivered raw barcoded gene expression/TCR data. The raw transcript data were normalized. Quality control (QC) steps were run on the normalized data to determine the appropriate level of cluster depth. T-SNE was performed on the transcriptomic data. The TCRs were projected onto a transcriptomic, t-SNE map.

For CITE-seq analyses, cryopreserved TIL were thawed and rested in the TIL medium without cytokine. The next day, dead cells were removed from TIL using the Dead Cell Removal Kit (Miltenyi Biotech, Bergisch Gladbach, Germany), and T-cells were further purified using the EASYSEP Human T Cell Isolation Kit (Stemcell Technologies, Vancouver, Canada). Next, T-cells were stained with a fluorochrome-labeled anti-CD3 antibody and feature-barcoding (FBC) antibodies including, but not limited to, anti-CD4, CD8a, CD45RA, CD45RO, CD62L, CD27, CD107a, HLA-DR, CD39, CD103, CD69, CD134, CD137, CD244, CD272, CD357, CD279, CD274, CD223, CD366, KLRG1, TIGIT, CD185 and CD278. Sony cell sorters (MA900 or SH800) were used to isolate CD3⁺ cells, and ˜50,000 T-cells were delivered to SCAF for the production of 10× single-cell libraries and deep-sequencing. Raw sequence data were processed by 10× Cell Ranger and Transcriptome, FBC, and TCR VDJ data were merged and analyzed by the 10× Loupe applications and PARTEK FLOW software.

### Example 1

This example demonstrates a method of isolating neoantigen-reactive TCRs from a human rectal cancer using single cell transcriptome analysis.

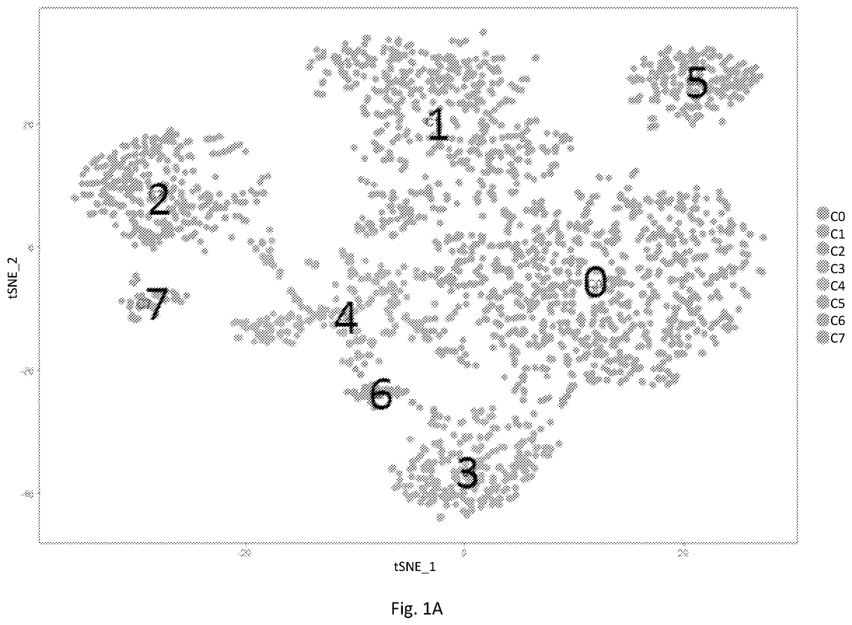

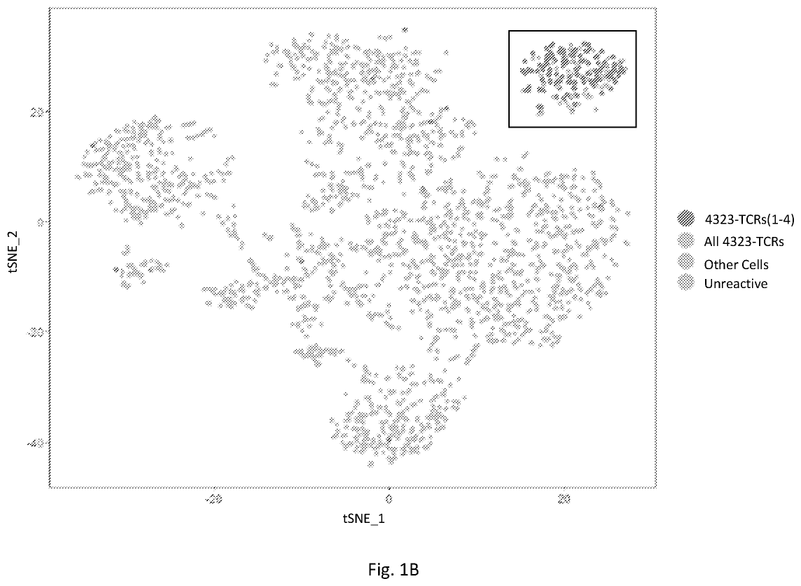

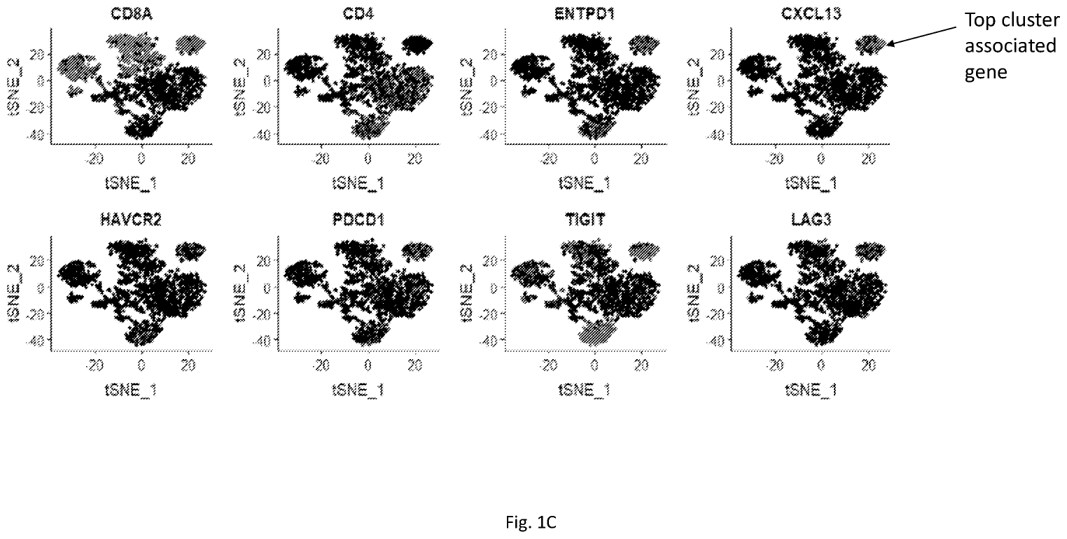

For the first time, autologous neoantigen-specific T-cells (molecularly defined for both mutated antigen and TCR sequence) were used to search for markers of T-cells with neoantigen reactivity. It is particularly notable that this was done using the TIL from common epithelial cancers such as colon and lung cancer. This was performed with both a transcriptomic approach as well as a barcoded antibody technique (CITE-seq). Single-cell suspensions were made by enzymatic digestion of fresh tumor specimens. A liver metastasis was harvested from a patient with rectal cancer (Patient 4323). For this patient, four (4) neoantigen-reactive CD8⁺ TCRs were previously identified using previously described techniques for screening TIL (Parkhurst et al., Cancer Discov., 9(8): 1022-1035 (2019)), totaling 6.6% of all TILs within the tumor. Flow cytometry was used to isolate CD4 positive and CD8 positive T cells from the tumor digest. 10×scTCR was performed at SCAF. T-SNE was performed on the transcriptomic data. The TCRs were projected onto a transcriptomic t-SNE map. The results are shown in FIGS. 1A-1C.

FIG. 1A shows the results of the t-SNE analysis of the T cells from Patient 4323. As shown in FIG. 1A, tSNE phenotypic clustering of the resulting single cell transcriptomic data showed that seven distinct phenotypic clusters were present within the sorted TIL (FIG. 1A; clusters numbered 0-7).

Known neoantigen-reactive TCRs were projected onto the t-SNE map of FIG. 1A. The results are shown in FIG. 1B. As shown in FIG. 1B, when the known neoantigen-reactive TCRs were overlaid onto the tSNE plots, almost all reactive TCRs localized to a single cluster, namely cluster 5. Cluster 5 was referred to as the neoantigen-reactive TCR (NeoTCR) cluster.

This NeoTCR cluster represented a dysfunctional CD8⁺ cell phenotype, as indicated by the presence of multiple activation/inhibitory markers, including CD39 (ENTPD1), PD-1 (PDCD1), TIGIT, CD69, LAG3, TIM3 (HAVCR2), CTLA4, and combinations thereof (FIG. 1C).

It was, therefore, hypothesized that other untested TCRs in this NeoTCR cluster might also be neoantigen-reactive. To test this hypothesis, the nine other TCRs in the NeoTCR cluster were prospectively reconstructed in silico using the single cell TCR sequencing data. Within cluster 5, 195 cells either expressed known neoantigen-reactive TCRs or had a TCR that could be in silico reconstructed.

The TCRs were cloned into pMSGV1 vectors, expressed in healthy donor PBL, and screened for reactivity against Patient 4323's dendritic cells (DCs) (i) electroporated with TMG encoding the patient's neoantigens or (ii) pulsed with pools of peptides encoding the patient's neoantigens. Seven o the nine new unknown TCRs (77.77%) were neoantigen-reactive in this screen.

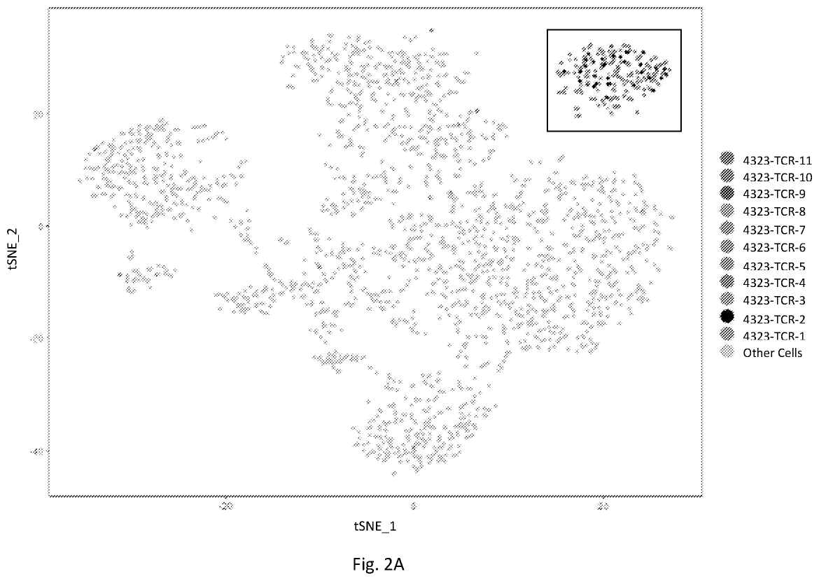

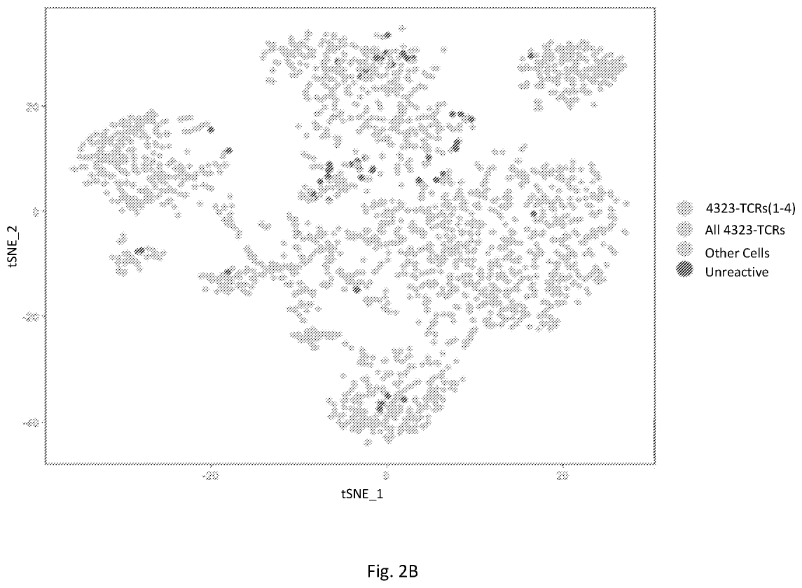

In total, 97% of the cells in cluster 5 were neoantigen-reactive (FIG. 2A). Some of the TCRs were rare enough to have been seen only one time by sequencing. In contrast, nonreactive clones (from this study and prior attempts to identify neoantigen-reactive TCRs for this patient) were identified in all eight clusters (FIG. 2B).

### Example 2

This example demonstrates that neoantigen reactivity is enriched within cell populations positive for multiple activation markers.

TIL harvested from Patient 4323 in Example 1 were cryopreserved. Cells were thawed and rested overnight without cytokines. Live CD3 cells were sorted into plates for single cell polymerase chain reaction (scPCR) and TCR reconstruction according to PD-1 (1 96-well plate), CD39 (1 96-well plate), TIGIT (0.5 plate), or LAG3 (0.5 plate) expression. The percentages of the sorted cells that were positive for expression of the markers were as follows: PD-1 (63.5%), CD39 (27.0%), TIGIT (31.1%), and LAG3 (0.74%). The sorted cells were sequenced by IMMUNOSEQ assay (Adaptive Biotechnologies). All 12 neoantigen-reactive TCRs could be analyzed for frequency among different populations.

A retrospective analysis of the adaptive sequencing of FACS-sorted populations was carried out. Table 1 shows the percentages of neoantigen-reactive TCRs within each population. The retrospective analysis showed enrichment of neoantigen reactivity within cell populations positive for multiple activation markers.

TABLE 1

| | Bulk | PD-1 | PD-1 | CD39 | CD39 | TIGIT | TIGIT | CD39/PD-1 | CD39/PD-1 | TIGIT/PD-1 | TIGIT/PD-1 |
|---|---|---|---|---|---|---|---|---|---|---|---|
| TCR ID | CD3⁺ | negative | positive | negative | positive | negative | positive | negative | positive | negative | positive |
| 1 | 3.21 | 0.48 | 13.54 | 0.00 | 19.88 | 0.00 | 14.13 | 0.00 | 19.92 | 0.00 | 17.12 |
| 2 | 2.03 | 0.10 | 9.81 | 0.04 | 10.74 | 0.15 | 8.37 | 0.00 | 12.50 | 0.00 | 13.23 |
| 5 | 1.56 | 0.00 | 8.15 | 0.04 | 10.04 | 0.10 | 8.76 | 0.00 | 14.84 | 0.00 | 11.67 |
| 3 | 1.30 | 0.00 | 6.22 | 0.00 | 9.64 | 0.05 | 7.26 | 0.00 | 16.41 | 0.00 | 10.89 |
| 8B1 | 0.18 | 0.00 | 0.97 | 0.00 | 1.19 | 0.05 | 0.79 | 0.00 | 0.39 | 0.00 | 0.78 |
| 12A2 | 0.16 | 0.00 | 0.41 | 0.00 | 0.40 | 0.00 | 0.08 | 0.00 | 0.78 | 0.00 | 1.17 |
| 4 | 0.09 | 0.00 | 0.28 | 0.00 | 0.80 | 0.05 | 0.08 | 0.12 | 0.00 | 0.00 | 0.39 |
| 6 | 0.09 | 0.00 | 0.28 | 0.00 | 0.50 | 0.00 | 0.71 | 0.00 | 0.39 | 0.00 | 0.78 |
| 9 | 0.08 | 0.00 | 0.00 | 0.00 | 0.10 | 0.00 | 0.08 | 0.00 | 0.00 | 0.00 | 0.39 |
| 7 | 0.05 | 0.00 | 0.00 | 0.00 | 0.30 | 0.00 | 0.32 | 0.00 | 1.17 | 0.00 | 0.00 |
| 8B2 | 0.07 | 0.00 | 0.14 | 0.00 | 0.00 | 0.00 | 0.16 | 0.00 | 0.00 | 0.00 | 0.00 |
| 10 | 0.02 | 0.10 | 0.14 | 0.00 | 0.20 | 0.00 | 0.16 | 0.00 | 0.00 | 0.00 | 0.39 |
| total | 8.83 | 0.67 | 39.92 | 0.07 | 53.78 | 0.41 | 40.88 | 0.12 | 66.41 | 0.00 | 56.81 |

### Example 3

This example demonstrates a method of isolating neoantigen-reactive TCRs from a human colon cancer using single cell transcriptome analysis.

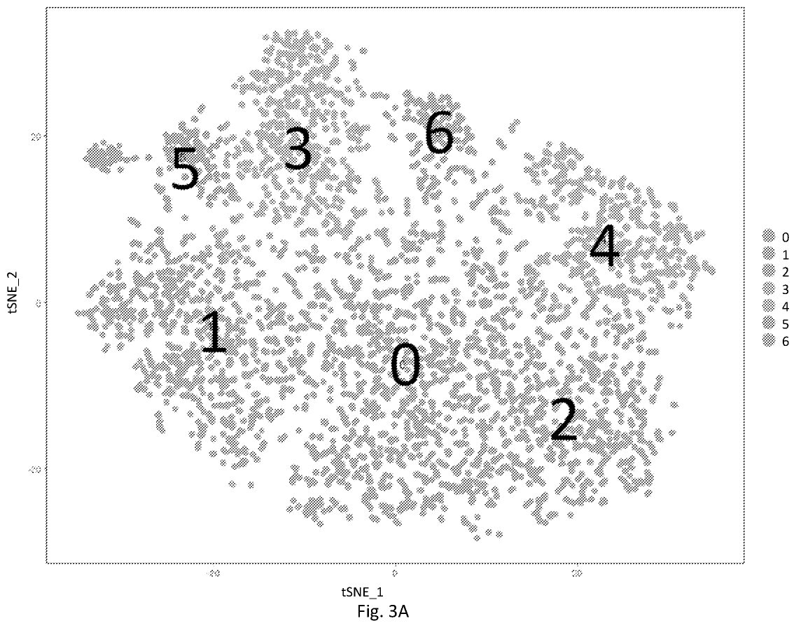

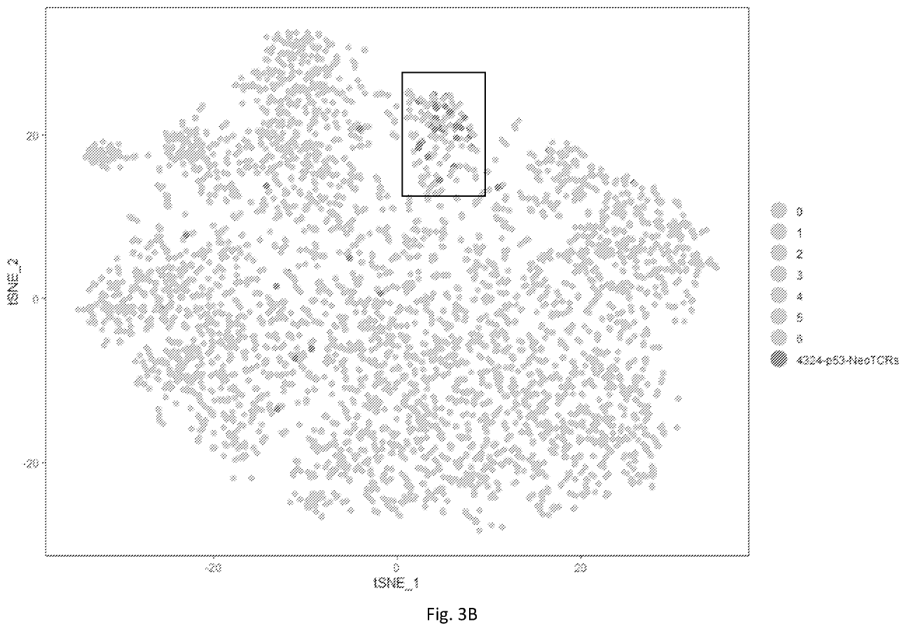

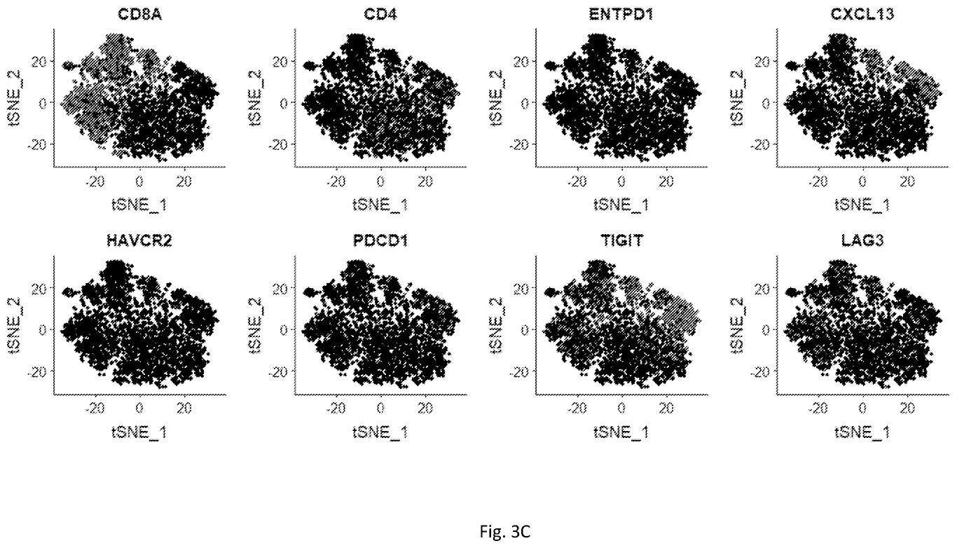

Single cell transcriptome analysis and TCR sequencing were performed on TIL that had been sorted from a lung metastasis that had been removed from a patient with colon cancer (Patient 4324). The results are shown in FIGS. 3A-3C. For this patient, three neoantigen-reactive CD8⁺ TCRs were previously identified, totaling 0.98% of all TILs within the tumor. These three TCRs recognized mutated TP53.

For the TIL from Patient 4324, not only were all known neoantigen-reactive CD8⁺ TCRs enriched within a single phenotypic cluster (namely, cluster 6) (FIG. 3B), but the cluster shared a number of markers with the NeoTCR cluster observed in sample 4323 (namely, the CD8⁺ markers listed in Table 2) (FIG. 3C). Further, there was an additional cluster (cluster 4) that contained CD4⁺ TIL that had similar phenotypes as the NeoTCR cluster.

Reconstruction of four TCRs from the NeoTCR cluster of 4324 yielded one with reactivity against mutated TP53. Four TCRs were reconstructed from the CD8⁺CXCL13⁺ cluster and tested against mutant TP53 (long peptide and tandem minigenes containing mutation-encoded amino acids). One TCR (namely, TCR number 5) was positive.

The markers common to the CD8⁺ NeoTCR cluster from 4323 and 4324 will be compiled into a CD8⁺ NeoTCR signature that can be applied to single cell transcriptome data to predict whether a TCR from a CD8⁺ cell will be cancer reactive. The same will be tested with a CD4⁺ NeoTCR signature.

Ten new TCRs were prospectively constructed from the CD8⁺ cluster. Fifteen new TCRs were prospectively constructed from the CD4⁺ cluster. It is intended to test whether these are neoantigen-reactive.

### Example 4

This example demonstrates that known CD4⁺ neoantigen-reactive TIL from breast cancer self-assemble into a phenotypic cluster marked by CXCL13 expression.

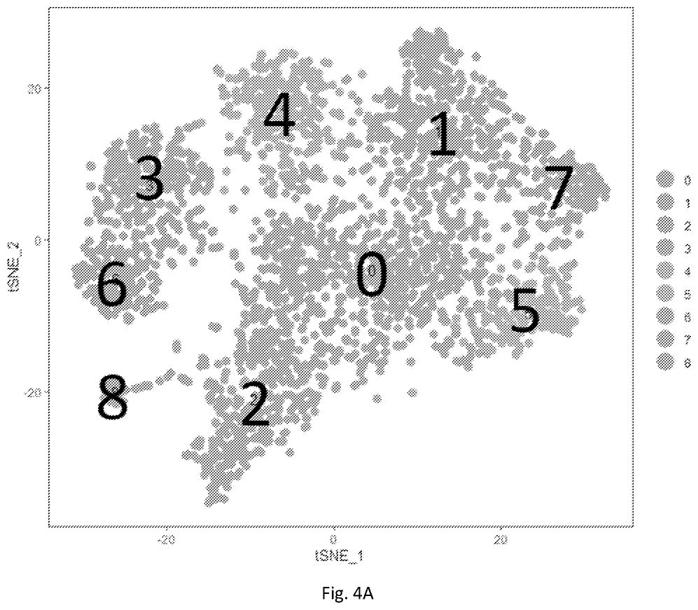

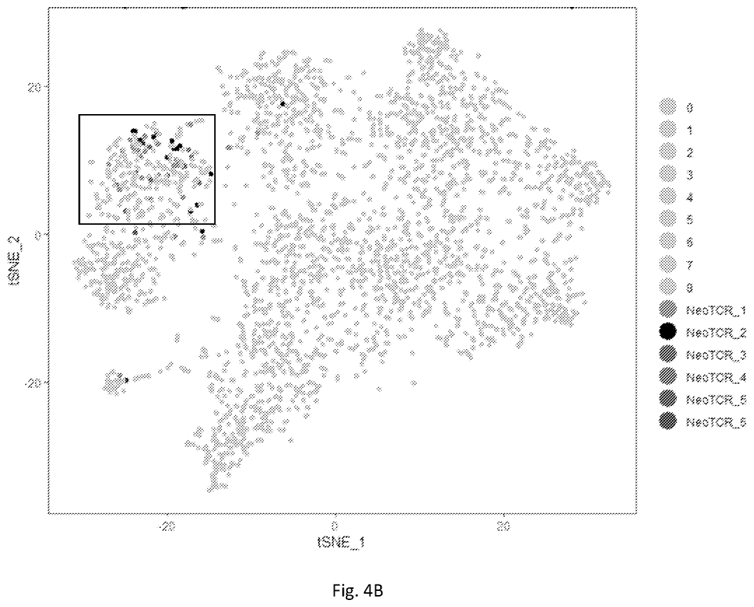

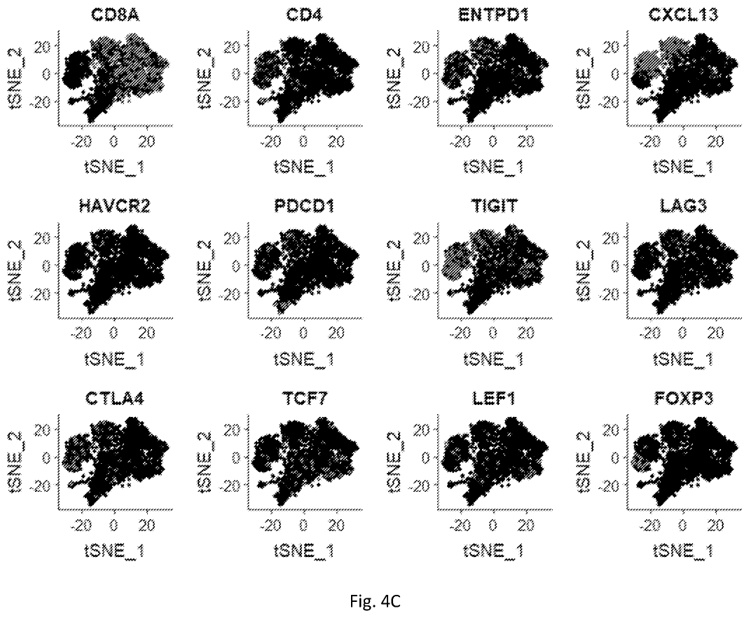

To test whether the neoantigen-reactive TCR signature would hold true in CD4⁺ TIL, single cell transcriptome and TCR sequencing were performed on TIL from a breast cancer metastasis sample (Patient 4322) in which six CD4⁺ neoantigen-reactive TCRs were known. The results are shown in FIGS. 4A-4C. In this sample, 2.4% of all TIL were known to be reactive (FIG. 4A).

All cells expressing the known CD4+NeoAg-reactive TCRs were found in a given cluster (namely, cluster 3) (boxed area of FIG. 4B), which expressed similar markers as the NeoTCR clusters in 4323 and 4324 (namely, the CD4⁺ markers listed in Table 2) (FIG. 4C), including CXCL13.

### Example 5

This example demonstrates that the CD8⁺ neoantigen-reactive TIL from lung cancer co-cluster with those from rectal cancer.

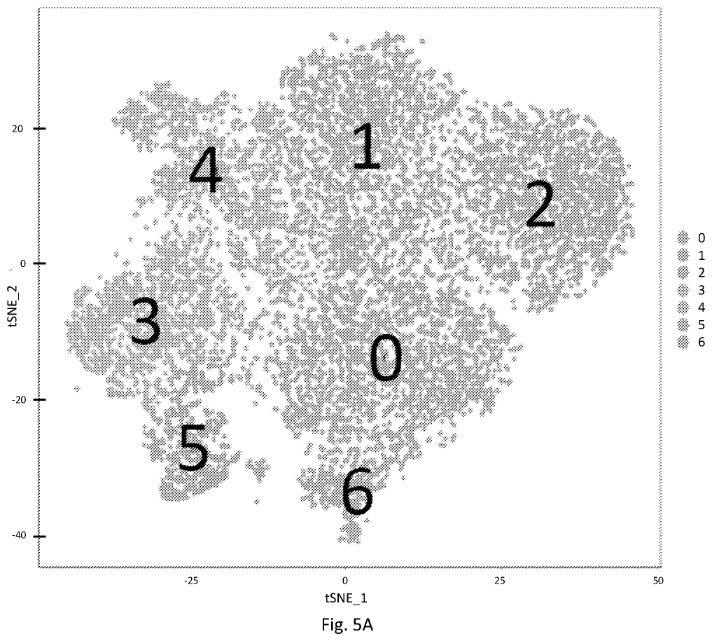

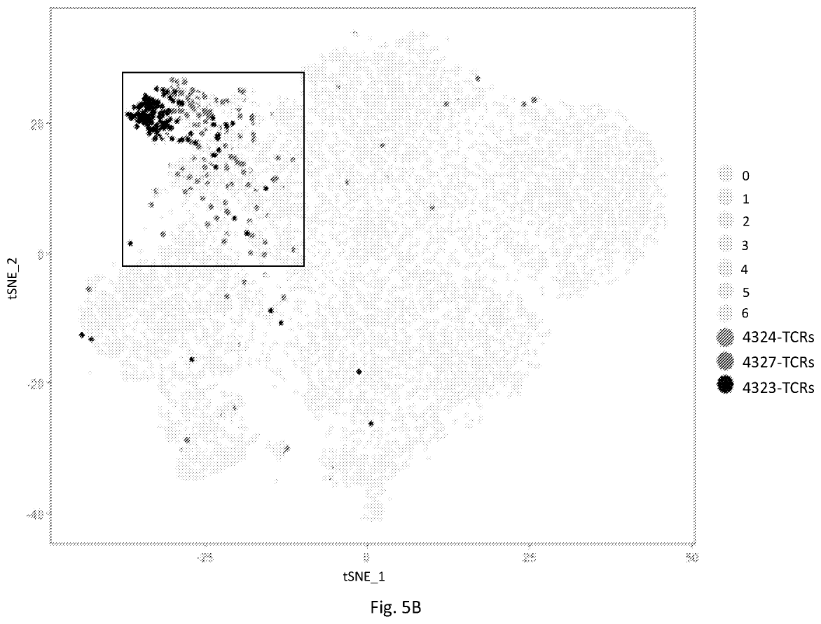

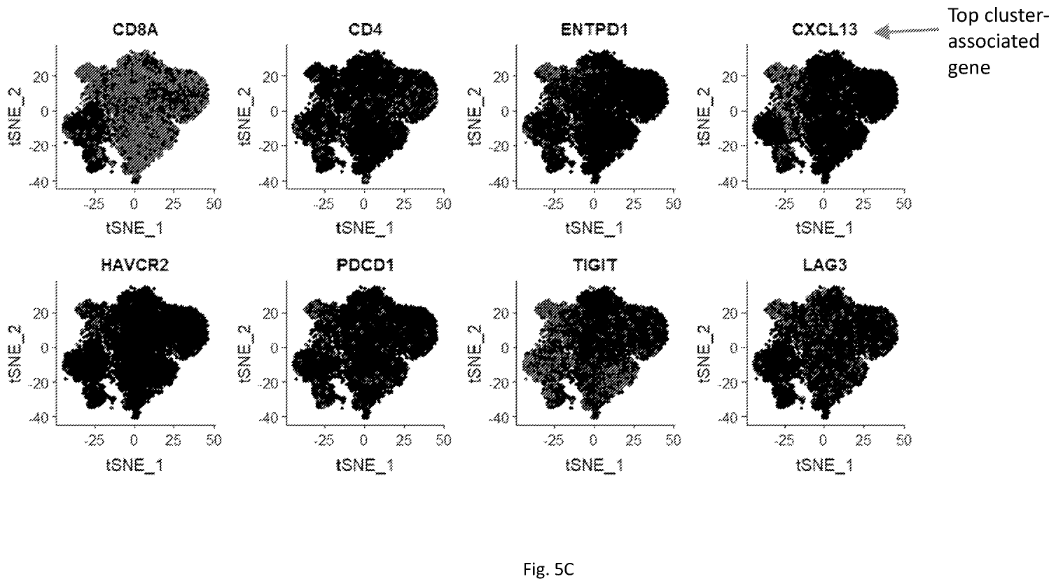

Single cell transcriptome/TCR sequencing had previously been carried out for TIL isolated from two surgically resected non-small cell lung cancer (NSCLC) tumors from which the TIL screens showed reactive TCRs (4234 & 4237, FIG. 5A).

Re-clustering of 4323 CD8⁺ clusters with these NSCLC samples showed that the reactive cells from all three samples were enriched in the same cluster (FIG. 5B).

This NeoTCR-containing cluster was positive for the same activation/exhaustion/checkpoint markers as the NeoTCR seen in the previous samples (FIG. 5C), indicating that the CD8⁺NeoTCR signature is not limited to TIL within gastrointestinal tumors, but is more broadly applicable to those infiltrating lung cancer as well.

### Example 6

This example demonstrates that known CD4⁺ neoantigen-reactive TIL from colon cancer self-assemble into a phenotypic cluster marked by CXCL13 expression.

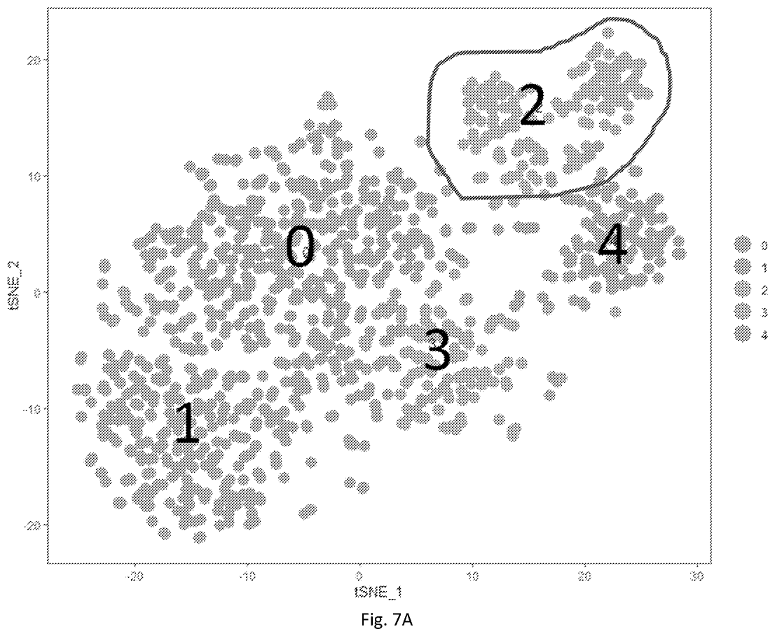

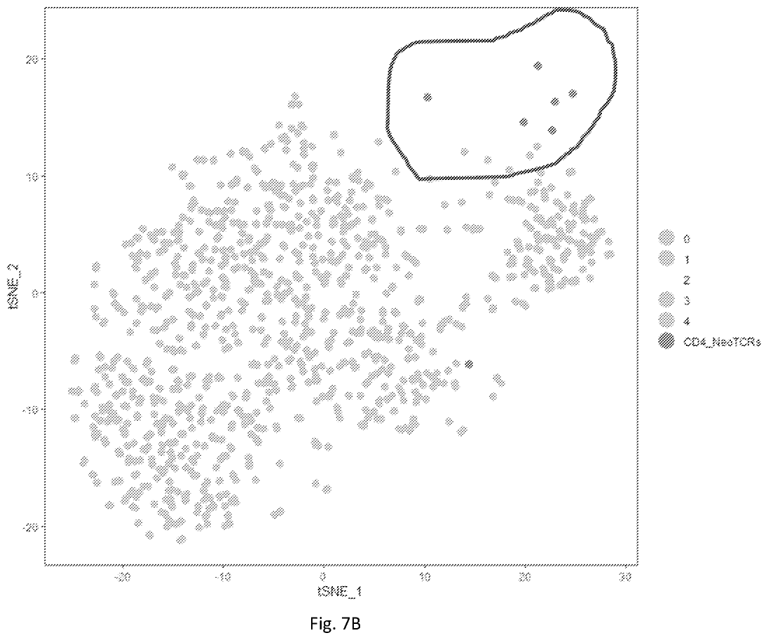

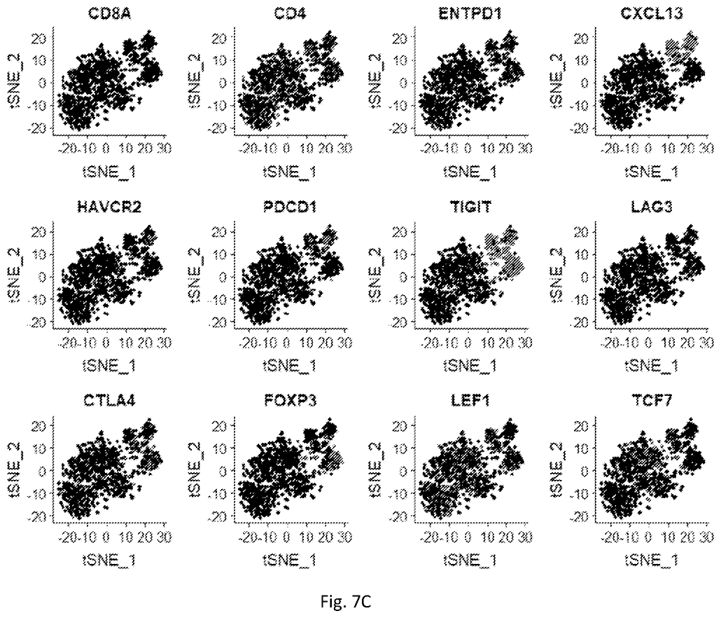

Single cell transcriptome and TCR sequencing were performed on TIL from a lung metastasis of colon cancer (Patient 4283) in which four CD4⁺ neoantigen-reactive TCRs were known. 10× sequencing captured three out of the four cells (only 6 total cells). The results are shown in FIGS. 7A-7C.

All cells expressing the known CD4⁺ NeoAg-reactive TCRs were found in a given cluster (namely, cluster 2) (FIG. 7B), which expressed CXCL13.

### Example 7

This example demonstrates that the markers set forth in Table 2 can be used to identify tumor mutation reactive T-cells from tumor digest with high confidence.

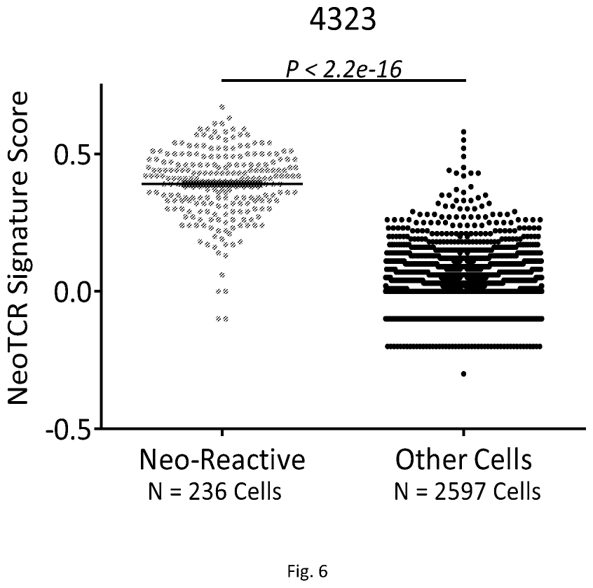

Using genes that are highly expressed in the NeoTCR cluster of 4323, a transcriptomic gene expression profile was developed for neoantigen-reactive TCRs termed “NeoTCR Signature.” Application of this signature to TILs from 4323 at the single cell level was able to clearly differentiate between known neoantigen reactive T cells and other cells (P<2×10⁻¹⁶, Wilcoxon rank-sum test) (FIG. 6). Thus, the NeoTCR signature can be prospectively used to score single T cells from a tumor. Based on high score of NeoTCR Signature, TCRs can be synthesized and tested for tumor reactivity.

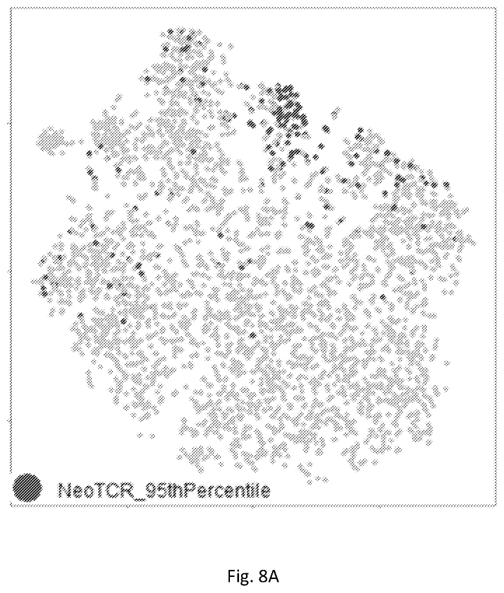

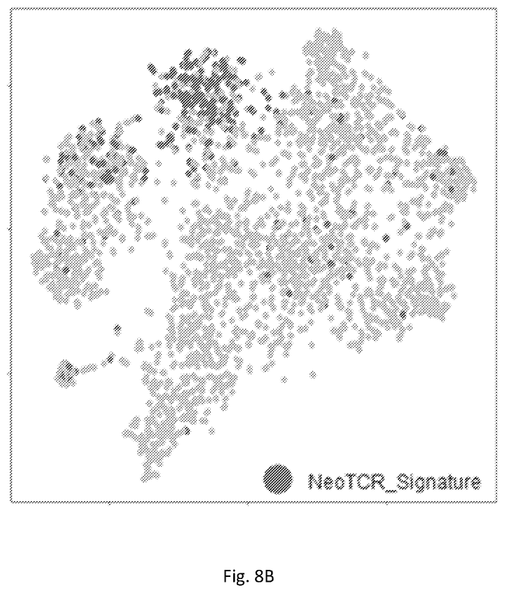

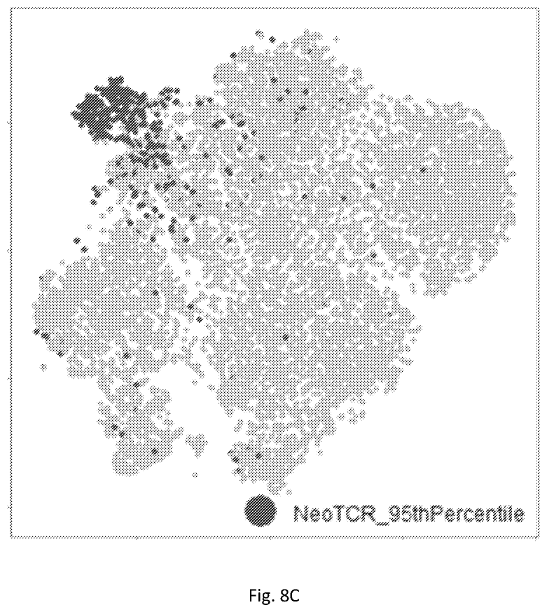

Using cells expressing the 95th percentile of NeoTCR signature derived from Pt.4323 (FIG. 6) onto the original tSNE plots of other patients showed that the NeoTCR signature identified the same cell clusters and cells with high confidence (FIGS. 3A-3C—Patient (Pt.) 4324; FIG. 4A-4C—Pt. 4322; and FIGS. 5A-5C—three patient samples, namely Patients 4323, 4237, and 4234). These results are summarized in FIGS. 8A-8C (8A-Patient 4324, 8B- Patient 4322, and 8C-Patients 4323, 4237, and 4234).

TABLE 2

| CD4⁺CD8⁺ Markers | CD4⁺ Markers | CD8⁺ Markers |
|---|---|---|
| CXCL13 | BATF | ALOX5AP |
| ITM2A | CD247 | ARHGAP9 |
| KLRB1 | CXCL13 | CARD16 |
| TIGIT | DNPH1 | CD3G |
| (−) LTB | DUSP4 | CD8A |
| (−) LYAR | GYPC | CD8B |
| (−) RGCC | IFITM1 | CLIC3 |
| (−) S100A10 | IGFLR1 | CTSW |
| | ITM2A | CXCL13 |
| | KLRB1 | CXCR6 |
| | LIMS1 | GALNT2 |
| | NMB | GZMB |
| | NR3C1 | HLA-DPA1 |
| | SH2D1A | HLA-DPB1 |
| | SPOCK2 | HLA-DRB1 |
| | SUPT3H | HLA-DRB5 |
| | TIGIT | HMGN3 |
| | TNFRSF18 | ITGAE |
| | (−) CCL5 | ITM2A |
| | (−) CD52 | KLRB1 |
| | (−) GSTP1 | MPST |
| | (−) JUN | NAP1L4 |
| | (−) LGALS1 | NELL2 |
| | (−) LTB | NSMCE1 |
| | (−) LYAR | PTMS |
| | (−) PLP2 | RAB27A |
| | (−) RGCC | RARRES3 |
| | (−) S100A10 | RBPJ |
| | (−) VIM | TIGIT |
| | (−) ZFP36 | (−) ANXA1 |
| | | (−) EEF1B2 |
| | | (−) EMP3 |
| | | (−) IL7R |
| | | (−) LGALS3 |
| | | (−) LTB |
| | | (−) LYAR |
| | | (−) RGCC |
| | | (−) RPL36A |
| | | (−) S100A10 |

Thus, markers listed in the NeoTCR signature shown in Table 2 can be used to identify tumor mutation reactive T-cells from tumor digest with high confidence. The first column of Table 2 lists the markers common to CD4⁺ and CD8⁺ neoantigen-reactive cells. The second column of Table 2 lists the markers common to CD4⁺ neoantigen-reactive cells. The third column of Table 2 lists the markers common to CD8⁺ neoantigen-reactive cells. The markers preceded by “(−)” in Table 2 are negatively associated with neoantigen reactivity. The markers which are not preceded by “(−)” in Table 2 are positively associated with neoantigen reactivity.

### Example 8

This example demonstrates a method of isolating neoantigen-reactive TCRs from a human rectal cancer using CITE-seq (Cellular Indexing of Transcriptomes and Epitopes by Sequencing) and antibodies.

CITE-seq is a single-cell analysis method that provides antibody-based cell surface molecule detection as well as TCR gene and transcriptome analysis. By using CITE- seq, it is possible to get more sensitive and quantitative cell-surface molecule expression data as compared to analysis of the transcriptome alone. For example, CITE-seq approach may be useful when the RNA quality of the tumor sample is compromised.

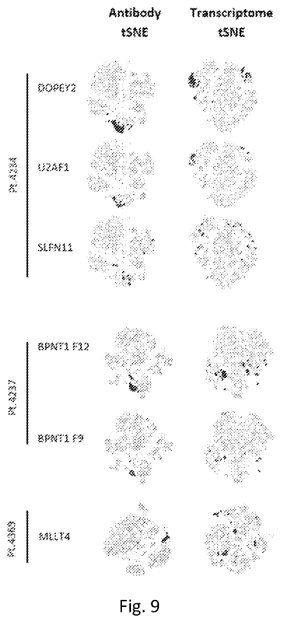

CITE-seq analysis was performed on three single-cell suspensions derived from Non-Small Cell Lung Cancer (NSCLC) specimens. First, the clustering of neoantigen-reactive CD8⁺ T-cells obtained by the CITE-seq-based tSNE and the transcriptome-based tSNE was compared (FIG. 9). As shown in FIG. 9, in most cases, the antibody-based tSNE plot resulted in better clustering of neoantigen-reactive T-cells.

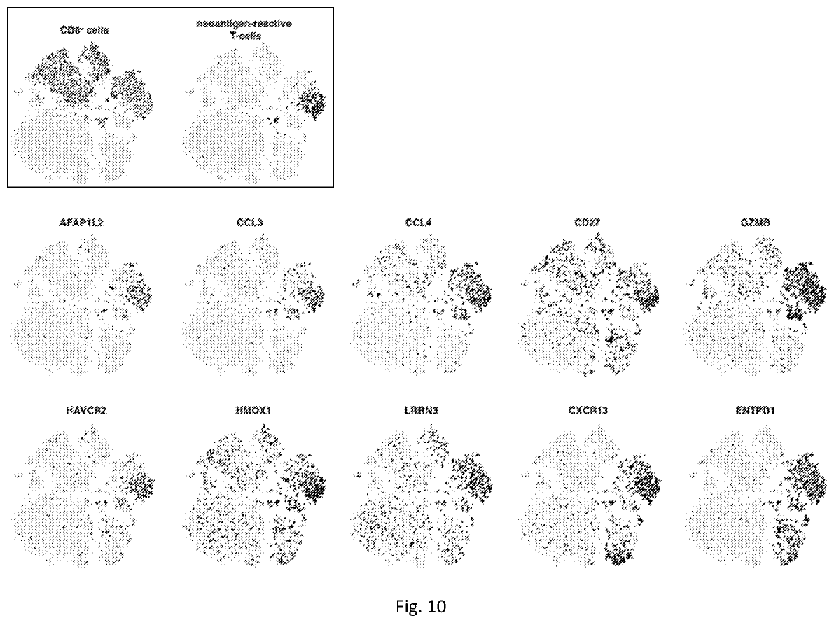

Next, which molecules were specifically expressed in neoantigen-reactive T-cells was examined. The results are shown in Tables 3-8 and FIG. 10.

TABLE 3
Patient 4234 (lung cancer analyzed by CITEseq)
DOPEY2-reactive CD8⁺ T-cells compared to other CD8⁺ T-cells
66 DOPEY2-reactive CD8⁺ T-cells were detected.
4682 other CD8⁺ cells were detected.

| Antibody-based | | | Transcriptome-based | | |
|---|---|---|---|---|---|
| | Log2 | | | Log2 | |
| | Fold | Adjusted | | Fold | Adjusted |
| | Change | p-value | | Change | p-value |
| PD-1⁺ | 1.71 | 1.63e−73 | TRAV25-2⁺ | 6.75 | 2.99e−24 |
| Tim-3⁺ | 1.48 | 8.39e−57 | TRBV5-6⁺ | 5.74 | 4.00e−18 |
| CD39⁺ | 1.96 | 5.38e−51 | CXCL13⁺ | 4.02 | 1.49e−6 |
| CD137⁺ | 0.26 | 2.04e−2 | HMGX1⁺ | 3.61 | 2.38e−4 |
| | | | GZMB⁺ | 2.72 | 2.34e−2 |
| | | | NKG7⁺ | 2.59 | 3.17e−2 |

TABLE 4
Patient 4234 (lung cancer analyzed by CITEseq)
U2AF1-reactive CD8⁺ T-cells compared to other CD8⁺ T-cells
15 U2AF1-reactive CD8⁺ T-cells were detected.
4259 other CD8⁺ cells were detected.

| | |
|---|---|
| Antibody-based | Transcriptome-based |

| | | | |
|---|---|---|---|
| Log2 | | Log2 | |
| Fold | Adjusted | Fold | Adjusted |
| Change | p-value | Change | p-value |

| | | | |
|---|---|---|---|
| CD39⁺ | 2.16 | 2.19E−15 | No significant differences |

| | | |
|---|---|---|
| PD-1⁺ | 1.51 | 1.36E−13 |
| Tim-3⁺ | 1.28 | 3.12E−11 |

TABLE 5
Patient 4234 (lung cancer analyzed by CITEseq)
SLFN11-reactive CD8⁺ T-cells compared to other CD8⁺ T-cells
15 SLFN11-reactive CD8⁺ T-cells were detected.
3366 other CD8⁺ cells were detected.

| Antibody-based | | | Transcriptome-based | | |
|---|---|---|---|---|---|
| | Log2 | | | Log2 | |
| | Fold | Adjusted | | Fold | Adjusted |
| | Change | p-value | | Change | p-value |
| CD39⁺ | 2.16 | 3.86E−67 | TRBV7-2⁺ | 7.00 | 1.00e−7 |
| CD103⁺ | 1.51 | 4.57e−1 | TRAV1-2⁺ | 4.87 | 1.94e−2 |
| PD-1⁺ | 1.28 | 2.90e−2 | | | |

TABLE 6
Patient 4237 (lung cancer analyzed by CITEseq)
MLLT4-reactive CD8⁺ T-cells compared to other CD8⁺ T-cells
43 MLLT4-reactive CD8⁺ T-cells were detected.
4350 other CD8⁺ cells were detected.

| Antibody-based | | | Transcriptome-based | | |
|---|---|---|---|---|---|
| | Log2 | | | Log2 | |
| | Fold | Adjusted | | Fold | Adjusted |
| | Change | p-value | | Change | p-value |
| CD39⁺ | 6.03 | 1.11e−106 | TRBV7-2⁺ | 6.19 | 4.28e−17 |
| CD137⁺ | 3.18 | 5.15e−35 | CXCL13⁺ | 4.65 | 1.86e−8 |
| Tim-3⁺ | 1.68 | 1.982e−11 | TRAV24⁺ | 4.69 | 1.78e−7 |
| | | | KRT86⁺ | 4.30 | 2.56e−5 |
| | | | HLA-DRA⁺ | 3.29 | 3.34e−4 |
| | | | HLA-DQA1⁺ | 3.34 | 5.83e−4 |
| | | | 4-1BB⁺ | 3.44 | 6.81e−4 |
| | | | GITR⁺ | 3.50 | 7.24e−4 |
| | | | HLA-DRB5⁺ | 3.10 | 1.90e−3 |
| | | | HLA-DQB1⁺ | 3.13 | 2.61e−3 |
| | | | HLA-DRB1⁺ | 2.96 | 2.67e−3 |
| | | | STMN1⁺ | 3.17 | 7.69e−3 |

TABLE 7
Patient 4237 (lung cancer analyzed by CITEseq)
BPNT1 F12-reactive CD8⁺ T-cells compared to other CD8⁺ T-cells
79 BPNT1 F12-reactive CD8⁺ T-cells were detected.
Other CD8⁺ cells were detected.

| Antibody-based | | | Transcriptome-based | | |
|---|---|---|---|---|---|
| | Log2 | | | Log2 | |
| | Fold | Adjusted | | Fold | Adjusted |
| | Change | p-value | | Change | p-value |
| CD39⁺ | 2.91 | 5.73e−101 | TRBV6-6⁺ | 7.62 | 5.18e−42 |
| PD-1⁺ | 1.96 | 1.01e−63 | CXCL13⁺ | 8.27 | 3.51e−35 |
| CD137⁺ | 1.82 | 8.14e−42 | TRAV25⁺ | 6.85 | 6.26e−26 |
| Tim-3⁺ | 1.47 | 3.80e−25 | ENTPD1⁺ | 4.38 | 8.03e−11 |
| CD134⁺ | 1.06 | 5.18e−13 | SLC1A4⁺ | 3.98 | 6.00e−10 |
| CCR7⁺ | 0.93 | 3.79e−12 | NSMCE1⁺ | 3.64 | 1.21e−8 |
| CD56⁺ | 0.96 | 4.90e−8 | CARS⁺ | 2.72 | 6.08e−4 |
| CD103⁺ | 0.61 | 4.85e−6 | CLIC3⁺ | 2.70 | 7.79e−4 |
| CD45RO⁺ | 0.52 | 1.88e−4 | HDLBP⁺ | 2.46 | 4.64e−3 |
| | | | GALNT2⁺ | 2.50 | 5.01e−3 |
| | | | TIGIT⁺ | 2.25 | 2.56e−2 |
| | | | DUSP4⁺ | 2.05 | 3.92e−2 |

TABLE 8
Patient 4237 (lung cancer analyzed by CITEseq)
BPNT1 F9-reactive CD8⁺ T-cells compared to other CD8⁺ T-cells
79 BPNT1 F9-reactive CD8⁺ T-cells were detected.
Other CD8⁺ cells were detected.

| Antibody-based | | | Transcriptome-based | | |
|---|---|---|---|---|---|
| | Log2 | | | Log2 | |
| | Fold | Adjusted | | Fold | Adjusted |
| | Change | p-value | | Change | p-value |
| CD39⁺ | 2.97 | 6.54e−16 | TRAV24⁺ | 8.38 | 1.43e−4 |
| PD-1⁺ | 2.12 | 5.00e−11 | CCNB1⁺ | 6.54 | 3.89e−4 |
| CD137⁺ | 1.44 | 1.72e−4 | CXCL13⁺ | 7.55 | 1.19e−2 |
| Tim-3⁺ | 1.32 | 8.31e−4 | TRBV5-1⁺ | 6.46 | 1.45e−2 |
| CD134⁺ | 0.95 | 2.17e−2 | PLK1⁺ | 6.33 | 1.45e−2 |
| CCR7⁺ | 0.74 | 4.79e−2 | | | |

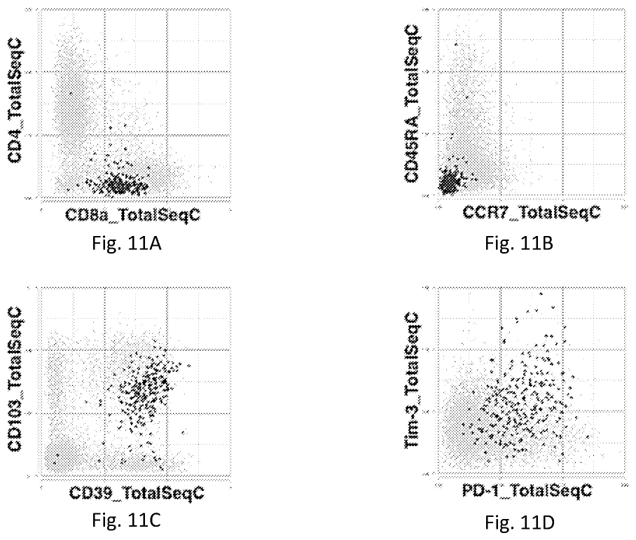

These analyses showed that neoantigen-reactive CD8+ T-cells expressed one or more of such cell surface molecules as CD27, CD39, CD74, CD103, CD106, CD137, HLA-DR, PD-1, Tim-3, and TIGIT. They were also marked by lower cell surface molecule expression of CCR7, CD8A, CD16, CD45RA, CD62L and IL7R as compared to other non-neoantigen-reactive CD8 cells (FIG. 11). As for intracellular molecules, in addition to the genes included in the NeoTCR signature described in Example 7, genes such as AFAP1IL2, ASB2, HMOX1, and PDLIM4 were expressed on neoantigen-reactive cells.

To test the hypothesis that this neoTCR signature could identify previously unknown TIL and TCRs that were mutation reactive, high-frequency clonotypes within the neoTCR-defined cluster were selected and their TCR genes were synthesized. These genes were introduced into PBL by retroviral transduction and subsequently co-cultured with dendritic cells that present neoantigen candidates that had been identified by the next generation sequencing of autologous tumors (Tables 9-11).

TABLE 9
Pt. 4234

| | %/CD3⁺ | TRAV | TRAJ | TRBV | YTBD | YRBJ | antigen |
|---|---|---|---|---|---|---|---|
| 1 | 0.61 | TRAV12-2 | TRAJ27 | TRBV30 | TRBD2 | TRBJ2-2 | DOPEY2 |
| 2 | 0.40 | TRAV8-4 | TRAJ47 | TRBV14 | | TRBJ1-1 | undetermined |
| 3 | 0.35 | TRAV8-3 | TRAJ20 | TRBV3-1 | TRBD2 | TRBJ1-2 | DOPEY2 |
| 4 | 0.35 | TRAV27 | TRAJ21 | TRBV6-1 | TRBD1 | TRBJ1-1 | PNPLA6 |
| 5 | 0.19 | TRAV8-3 | TRAJ20 | TRBV19 | TRBD1 | TRBJ2-1 | DOPEY2 |

For Patient 4234, out of five previously unknown TCR clonotypes interrogated, four of them were neoantigen-reactive. Remarkably, all of them existed at less than 1% in CD3⁺ cells, and the PNPLA6 reactivity had not been identified by any traditional TIL screening method.

TABLE 10
Pt. 4237

| TCR ID | %/CD3⁺ | TRAV | TRAJ | TRBV | TRBD | TRBJ | Antigen |
|---|---|---|---|---|---|---|---|
| F12 | 0.69 | TRAV25 | TRAJ54 | TRBV6-6 | TRBD2 | TRBJ2-1 | BPNT1 |
| 1 | 0.58 | TRAV4 | TRAJ40 | TRBV9 | | TRBJ2-3 | |
| 2 | 0.1 | TRAV8-1 | TRAJ39 | TRBV7-6 | TRBD2 | TRBJ2-3 | BPNT1 |
| 3 | 0.1 | TRAV8-6 | TRAJ39 | TRBV7-6 | TRBD2 | TRBJ2-1 | BPNT1 |
| F9 | 0.08 | TRAV24 | TRAJ29 | TRBV5-1 | TRBD1 | TRBJ1-1 | BPNT1 |
| 4 | 0.06 | TRAV1-2 | TRAJ20 | TRBV20-1 | TRBD1 | TRBJ1-2 | BPNT1 |
| 5 | 0.02 | TRAV29DV5 | TRAJ43 | TRBV12-5 | TRBD1 | TRBJ2-7 | |

For Patient 4237, TCRs F12 and F9 were identified by traditional TIL screening methods but are high-frequency clonotypes ranking the first and the fourth in the cluster. Out of five other undefined TCR clonotypes selected by neoTCR clustering, three of them proved to also recognize the BPNT1 neoantigen. In total, five out of the six most frequent TCR clonotypes residing in the neoTCR cluster were specifically reactive to mutated BPNT1.

TABLE 11
Patient 4369

| TCR ID | %/CD3⁺ | TRAV | TRAJ | TRBV | TRBD | TRBJ | Antigen |
|---|---|---|---|---|---|---|---|
| MLLT4 | 0.46 | TRAV24 | TRAJ53 | TRBV7-2 | | TRBJ2-3 | MLLT4 |
| 1 | 0.13 | TRAV29DV5 | TRAJ37 | TRBV7-2 | TRBD1 | TRBJ2-7 | |
| 2 | 0.09 | TRAV24 | TRAJ53 | TRBV5-5 | TRBD1 | TRBJ2-5 | MLLT4 |
| 3 | 0.07 | TRAV13-1 | TRAJ42 | TRBV2 | TRBD2 | TRBJ2-1 | |
| 4 | 0.07 | TRAV3 | TRAJ30 | TRBV6-5 | TRBD2 | TRBJ1-5 | |
| 5 | 0.06 | TRAV21 | TRAJ39 | TRBV2 | TRBD1 | TRBJ2-7 | MLLT4 |

For Patient 4369, the top frequency clonotype was identified by the traditional TIL screening. Out of five additional unknown clonotypes selected by frequency, two of them were reactive to mutated MLLT4. These two new MLLT4-reactive clonotypes existed at lower than 0.1% of the total TIL population by TCR sequencing. This shows the potential of this method in selecting neoantigen-reactive T-cells. It is possible that other high-frequency clonotypes within this cluster may recognize other as-yet-unidentified tumor-associated antigens such as the cancer-germline family of antigens.

### Example 9

This example demonstrates that sorting for PD-1⁺/CD39⁺/TGIT⁺ cells can enrich neoantigen-reactive CD8⁺ cells to a high degree.

Two plates of cells from Patient 4323 were sorted for expression of CD8, PD-1, CD39, and TIGIT using FACS. The cells were gated through live CD3⁺CD8⁺. Out of 140 legible TCR beta chain sequences, 123 were known to be neoantigen-reactive TCRs (88%) (Table 12).

TABLE 12

| | | CD8⁺ | |
|---|---|---|---|
| TCR ID | Bulk CD3⁺ (from Adaptive) | PD-1⁺/CD39⁺/TIGIT⁺ | |
| 1 | 3.21 | 29.2 | (41/140) |
| 2 | 2.03 | 24.2 | (34/140) |
| 5 | 1.56 | 15.0 | (21/140) |
| 3 | 1.30 | 12.1 | (17/140) |
| 8B1 | 0.18 | 3.6 | (5/140) |
| 12A2 | 0.16 | 0 | |
| 4 | 0.09 | 0.7 | (1/140) |
| 6 | 0.09 | 0.7 | (1/140) |
| 9 | 0.08 | 0.7 | (1/140) |
| 7 | 0.05 | 0 | |
| 8B2 | 0.07 | 1.4 | (2/140) |
| 10 | 0.02 | 0.7 | (1/140) |

| | | |
|---|---|---|
| total known reactive | 8.83 | 87.9 |

### Example 10

This example demonstrates that CXCL13⁺ capture results in a similar enrichment of known neoantigen-reactive CD8⁺ cells from Patient 4323 as PD-1⁺/CD39⁺/TIGIT⁺.

No off-the shelf CXCL13 capture reagents were available, but CXCL13 is reported to be detectable in vitro by ELISA without specific stimulation/activation. A biotinylated anti-CXCL13 monoclonal antibody was bound to an off-the-shelf CD45-streptavidin conjugate. A 4323 tumor digest was thawed and incubated overnight or for four hours with CD45-streptavidin:CXCL13 biotin in-house capture antibody. The sample was then washed and incubated with either goat IgG or goat anti-CXCL13 secondary antibody, and an anti-goat IgG PE-conjugated detection antibody. The sample was run on the cell sorter (Sony MA900). CD8⁺CXCL13⁺ cells (33) were sorted for scPCR TCR sequencing. Of the 33 cells sorted, 28 legible CDR3 beta sequences were identified. Out of 28 legible TCR beta chain sequences, 85.7% were known to be neoantigen-reactive TCRs (Table 13). Sorting based on CXCL13 expression may avoid the problem of not having an ideal set of surface markers for neaontigen-reactive CD4+ cells.

TABLE 13

| | | CD8⁺ | | | |
|---|---|---|---|---|---|
| | Bulk CD3⁺ | PD-1⁺/ | | | |
| | (from | CD39⁺/ | | CXCL13⁺ | |
| TCR ID | Adaptive) | TIGIT⁺ | | capture | |
| 1 | 3.21 | 29.2 | (41/140) | 25.0 | (7/28) |
| 2 | 2.03 | 24.2 | (34/140) | 14.3 | (4/28) |
| 5 | 1.56 | 15.0 | (21/140) | 28.6 | (8/28) |
| 3 | 1.30 | 12.1 | (17/140) | 10.7 | (3/28) |
| 8B1 | 0.18 | 3.6 | (5/140) | 3.6 | (1/28) |
| 12A2 | 0.16 | 0 | | 0 | |
| 4 | 0.09 | 0.7 | (1/140) | 3.6 | (1/28) |

| 6 | 0.09 | 0.7 | (1/140) | 0 |
|---|---|---|---|---|
| 9 | 0.08 | 0.7 | (1/140) | 0 |
| 7 | 0.05 | 0 | | 0 |
| 8B2 | 0.07 | 1.4 | (2/140) | 0 |
| 10 | 0.02 | 0.7 | (1/140) | 0 |

| | | | |
|---|---|---|---|
| total known reactive | 8.83 | 87.9 | 85.7 |

### Example 11

This example demonstrates that a CXCL13 expression assay can identify the coexpressed markers indicating neoantigen reactivity.

Patient 4397 underwent a mestastatic anal cancer TIL harvest. A tumor digest was made. Cells were immediately stained with CD45:CXCL13 bispecific antibody overnight. Cells were stained for CXCL13 and PD-1, CD39, and TIGIT and gated through live CD³⁺. CD4+CXCL13+ cells were the highest in frequency in CD39⁺/TIGIT⁺/PD-1⁻ cells (Table 14). CD8⁺ CXCK13⁺ were highest in frequency in CD39⁺/TIGIT⁺/PD-1⁺ cells (Table 14).

TABLE 14

| | | CD4⁺ through | | CD8⁺ through |
|---|---|---|---|---|
| Surface Markers | CD4⁺ | CXCL13⁺ | CD8⁺ | CXCL13⁺ |
| NONE | 45.07 | 2.11 | 18.35 | 4.36 |
| PD1⁺ alone | 2.48 | 2.11 | 1.98 | 0.00 |
| CD39⁺ alone | 6.91 | 4.21 | 3.22 | 2.18 |
| TIGIT⁺ alone | 14.60 | 5.27 | 19.46 | 2.17 |
| PD1⁺/CD39⁺ | 1.76 | 0.00 | 2.85 | 4.36 |
| PD1⁺/TIGIT⁺ | 4.43 | 11.54 | 9.66 | 4.35 |
| CD39⁺/TIGIT⁺ | 17.34 | 44.24 | 15.18 | 21.74 |
| PD1⁺/CD39⁺/TIGIT⁺ | 5.87 | 30.50 | 24.77 | 60.86 |

### Example 12

This example demonstrates a workflow for rapid neo-antigen TCR isolation from tumors using single cell analysis.

As shown in Examples 1-11, using clonally defined T-cells from common epithelial cancers (colorectal and lung), a signature of T-cells that specifically recognize tumor-associated mutated antigens (neoantigens) was identified. This was done with both a single cell transcriptome-based approach and using barcoded antibodies (CITE-seq) and it could cluster such cells within a narrowly defined space on multidimensional (tSNE) plots.

Using this neoTCR signature, other cells with this same phenotype that co-clustered with the known neoantigen-reactive T-cells were interrogated and found to contain a very high frequency of previously-unknown T-cell clones also recognizing neoantigens from the same tumor.

This technique not only expanded the repertoire of T-cells recognizing a known neoantigen, but could identify T-cells with specificity for a new neoantigen not identified as immunogenic by any other conventional screening methods.

The high sensitivity and specificity of this approach and the feature that it is performed directly from the TIL of a fresh tumor specimen distinguishes it from conventional methods of finding mutation-reactive T-cells.

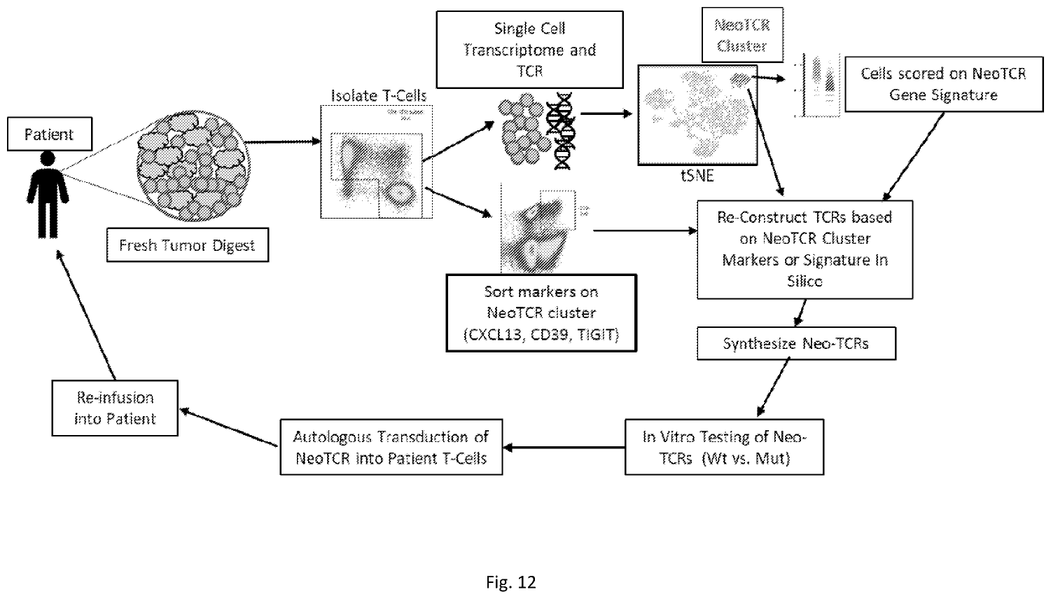

The ability to rapidly determine the sequence of the reactive TCRs is also of great value in the translation of this information into TCR-engineered T-cell populations for therapy. Using the data accrued from these several patients outlined in Examples 1-11, a workflow was designed for rapid TCR isolation from human tumors regardless of the histology of the tumor. This workflow is outlined in FIG. 12.

### Example 13

This example demonstrates the prospective isolation of an HPV 16-reactive TCR from a fresh tumor resection.

T cells from Patient 4397 (anal cancer) were sorted by PD-1, CD39, and TIGIT co-expression and subjected to TCR sequencing. The top 11 TCRs seen within this population were tested against patient neoantigens and HPV16 antigens, as the resected tumor specimen showed expression of HPV16 E4. Table 15 summarizes the top 11 TCRs within the CD39⁺PD1⁺TIGIT⁺ sorted population, with TCR1 highlighted. The numbers in Table 15 refer to percentages within bulk and enriched populations.

TABLE 15

| | | CD3⁺ | |
|---|---|---|---|
| TCR ID | Bulk CD3 | PD-1⁺/CD39⁺/TIGIT⁺ | |
| 1 | 0.2 | 7.5 | (12/159) |
| 2 | 0.0 | 6.3 | (10/159) |
| 3 | 0.0 | 6.3 | (10/159) |
| 5 | 0.6 | 3.1 | (5/159) |
| 7 | 0.0 | 1.9 | (3/159) |
| 6 | 0.0 | 1.9 | (3/159) |
| 4 | 0.0 | 1.9 | (3/159) |
| 8 | 0.2 | 1.9 | (3/159) |
| 9 | 0.0 | 1.3 | (2/159) |
| 12 | 0.0 | 1.3 | (2/159) |
| 10 | 0.0 | 1.3 | (2/159) |

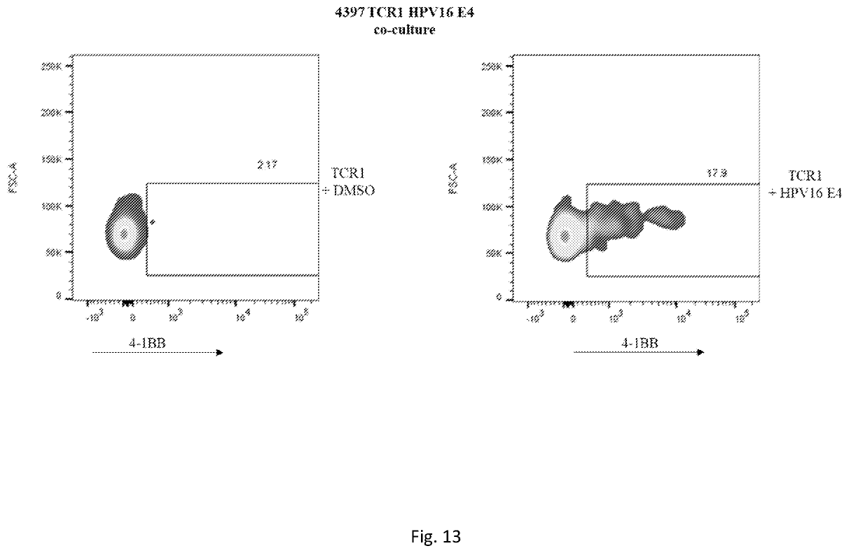

Screening of each of the 11 TCRs of Table 15 against HPV16-derived peptides showed reactivity against HPV16 E4 by TCR ID 1 (TCR1) (FIG. 13). Further testing of TCR1 showed reactivity against CD8-restricted HPV16 E4 minimal epitope LQSSLHLTA (SEQ ID NO: 1) presented by HLA-B*13:02.

### Example 14

This example demonstrates a method of isolating neoantigen-reactive TCRs from human cancer using single cell transcriptome analysis.

The gene expression profiles for identifying neoantigen reactive T cell receptors (TCRs) was further refined as follows. Over 45,000 tumor infiltrating T cells from over 13 patient samples spanning multiple tumor types and histologies were analyzed by single cell transcriptome analysis as described in Example 1. The gene expression profiles were consistently validated successfully in all of these patient T cells. The gene expression profiles of neoantigen reactive T cells for CD4 as well as CD8, in addition to common genes, are set forth in Table 16.

TABLE 16

| NeoTCR+ | CD4⁺ Markers | CD8⁺ Markers | CD4⁺CD8⁺ Markers |
|---|---|---|---|
| ABI3+ | ADI1+ | AC243829.4+ | AHI1+ |
| AC243960.1+ | AHI1+ | ACP5+ | CXCL13+ |
| ACP5+ | ARID5B+ | APOBEC3C+ | FABP5+ |
| ADGRG1+ | BATF+ | CCL3+ | NAP1L4+ |
| AHI1+ | CD4+ | CCL4+ | ORMDL3+ |
| ASB2+ | CMTM7+ | CCL4L2+ | PPP1R16B+ |
| BST2+ | CPM+ | CCL5+ | SH2D1A+ |
| CARS+ | CXCL13+ | CD27+ | TIGIT+ |
| CCL4+ | CYTH1+ | CD8A+ | TOX+ |
| CD27+ | ELMO1+ | CD8B+ | |
| CD2BP2+ | ETV7+ | CST7+ | |
| CD82+ | FABP5+ | CTSW+ | |
| CTSW+ | FBLN7+ | CXCL13+ | |
| CXCL13+ | FKBP5+ | DUSP4+ | |
| CXCR6+ | GRAMD1A+ | ENTPD1+ | |
| DUSP4+ | HIF1A+ | FABP5+ | |
| ENTPD1+ | IL6ST+ | GALNT2+ | |
| GALNT2+ | ITGA4+ | GNLY+ | |
| GATA3+ | ITK+ | GZMA+ | |
| GPR25+ | JAK3+ | GZMB+ | |
| GZMB+ | KLRB1+ | GZMH+ | |
| HDLBP+ | LEF1+ | GZMK+ | |
| HLA-DPA1+ | LIMS1+ | HAVCR2+ | |
| HLA-DRB1+ | MAF+ | HCST+ | |
| HMOX1+ | MAL+ | HLA-DMA+ | |
| ID2+ | MIR4435-2HG+ | HLA-DPA1+ | |
| IGFLR1+ | MYL6B+ | HLA-DPB1+ | |
| ITGAL+ | NAP1L4+ | HLA-DRA+ | |
| LAG3+ | NMB+ | HLA-DRB1+ | |
| LINC01871+ | NR3C1+ | HLA-DRB5+ | |
| LINC01943+ | PASK+ | HMOX1+ | |
| MIS18BP1+ | PGM2L1+ | IFNG+ | |
| MPST+ | PIM2+ | IGFLR1+ | |
| NCF4+ | PPP1CC+ | ITGAL+ | |
| NSMCE1+ | SESN3+ | JAML+ | |
| PCED1B+ | SH2D1A+ | LAG3+ | |
| PDCD1+ | SOCS1+ | LINC01871+ | |
| PHPT1+ | STAT1+ | LYST+ | |
| PLEKHF1+ | SYNE2+ | MIR155HG+ | |
| PRF1+ | TBC1D4+ | NKG7+ | |
| PTMS+ | TIGIT+ | PLEKHF1+ | |
| SLC1A4+ | TLK1+ | PRF1+ | |
| SLF1+ | TMEM123+ | PTMS+ | |
| SMC4+ | TMEM70+ | RGS1+ | |
| SUPT3H+ | TNIK+ | SLF1+ | |
| TIGIT+ | TOX+ | SMC4+ | |
| TNFRSF18+ | TSHZ2+ | SUPT3H+ | |
| TOX+ | UCP2+ | TIGIT+ | |
| TRAF3IP3+ | VOPP1+ | TOX+ | |
| YPEL2+ | YPEL2+ | | |

The NeoTCR gene signature was further evaluated for identifying mutation reactive T cells in blinded prospective patient tumor samples. TCRs reconstructed from single cell transcrptome sequencing and application of the NeoTCR signature yielded novel CD4 and CD8 NeoTCRs. Altogether, this study provided successful enrichment and detection of tumor-specific NeoTCRs in the sequenced TIL of 12/12 patients for whom reactivity was identified. The NeoTCR gene signature is also distinct from irrelevant viral-specific T cells and can, thus, accurately discriminate tumor-irrelevant T cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing an enriched population of T cells having antigenic specificity for a target antigen, the method comprising:
isolating T cells from a tumor sample of a patient;
selecting the isolated T cells which have a gene expression profile; and
separating the selected T cells from the unselected cells, wherein the separated selected T cells provide an enriched population of T cells having antigenic specificity for the target antigen,
wherein the target antigen is a neoantigen encoded by a cancer-specific mutation, a cancer antigen, or a cancer-associated viral antigen, and the gene expression profile comprises:
(a) (i) one or both of CD4⁺ and CD8⁺ and (ii) one or more of AFAP1IL2⁺, ASB2⁺, CXCL13⁺, HMOX1⁺, ITM2A⁺, KLRB1⁺, PDLIM4⁺, TGIT⁺, LTB⁻, LYAR⁻, RGCC⁻, and S100A10⁻;
(b) CD4⁺ and one or more of BATF⁺, CD247⁺, CXCL13⁺, DNPH1⁺, DUSP4⁺, GYPC⁺, IFITM1⁺, IGFLR1⁺, ITM2A⁺, KLRB1⁺, LIMS1⁺, NMB⁺, NR3C1⁺, SH2D1A⁺, SPOCK2⁺, SUPT3H⁺, TIGIT⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻;
(c) CD8⁺ and one or more of AFAP1IL2⁺, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, TIGIT⁺, ANXA1⁻, EEF1B2⁻, EMP3⁻, IL7R⁻, LGALS3⁻, LTB⁻, LYAR⁻, RGCC⁻, RPL36A⁻, and S100A10⁻;
(d) CD8⁺ and one or more of CD39⁺, CD74⁺, CD103⁺, CD106⁺, CD137⁺, HLA-DR⁺, TGIT⁺, CCR7⁻, CD8A⁻, CD16⁻, CD45RA⁻, CD62L⁻ and IL7R⁻;
(e) one or more of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺SMC4⁺, SUPT3H⁺, TIGIT⁺, TNFRSF18⁺, TOX⁺, TRAF3IP3⁺, and YPEL2⁺;
(f) CD4⁺ and one or more of ADI1⁺, AHI1⁺, ARID5B⁺, BATF⁺, CMTM7⁺, CPM⁺, CXCL13⁺, CYTH1⁺, ELMO1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TIGIT⁺, TLK1⁺, TMEM123⁺, TMEM70⁺, TNIK⁺, TOX⁺, TSHZ2⁺, UCP2⁺, VOPP1⁺, and YPEL2⁺;
(g) CD8⁺ and one or more of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺;
(h) one or more of AHI1+, CXCL13+, FABP5+, NAP1L4+, ORMDL3+, PPP1R16B+, SH2D1A+, TIGIT+, and TOX+; or
(i) one or more of TIGIT⁺, CD39⁺, and PD-1⁺.

2. A method of isolating a T cell receptor (TCR), or an antigen-binding portion thereof, having antigenic specificity for a target antigen, the method comprising:
preparing an enriched population of T cells having antigenic specificity for the target antigen according to the method of claim 1;
sorting the T cells in the enriched population into separate single T cell samples;
sequencing TCR complementarity determining regions 3 (CDR3) in one or more of the separate single T cell samples;
pairing an alpha chain variable region comprising a CDR3 with a beta chain variable region comprising a CDR3 encoded by the nucleic acid of the separate single T cell samples;
introducing a nucleotide sequence encoding the paired alpha chain variable region and beta chain variable region into host cells and expressing the paired alpha chain variable region and beta chain variable region by the host cells;
screening the host cells expressing the paired alpha chain variable region and beta chain variable region for antigenic specificity for the target antigen; and
selecting the paired alpha chain variable region and beta chain variable region that have antigenic specificity for the target antigen,
wherein the TCR, or an antigen-binding portion thereof, having antigenic specificity for the target antigen is isolated.

3. A method of isolating a T cell receptor (TCR), or an antigen-binding portion thereof, having antigenic specificity for a target antigen, the method comprising:
isolating T cells from a tumor sample of a patient;
sorting the T cells in the enriched population into separate single T cell samples;
sequencing TCR complementarity determining regions 3 (CDR3) in the separate single T cell samples;
selecting the separate single T cell samples which have a gene expression profile;
pairing an alpha chain variable region comprising a CDR3 with a beta chain variable region comprising a CDR3 encoded by the nucleic acid of the separate single T cell samples with the gene expression profile;
introducing a nucleotide sequence encoding the paired alpha chain variable region and beta chain variable region into host cells and expressing the paired alpha chain variable region and beta chain variable region by the host cells;
screening the host cells expressing the paired alpha chain variable region and beta chain variable region for antigenic specificity for the target antigen; and
selecting the paired alpha chain variable region and beta chain variable region that have antigenic specificity for the target antigen,
wherein the TCR, or an antigen-binding portion thereof, having antigenic specificity for the target antigen is isolated,
wherein the target antigen is a neoantigen encoded by a cancer-specific mutation, a cancer antigen, or a cancer-associated viral antigen and the gene expression profile comprises:
(a) (i) one or both of CD4⁺ and CD8⁺ and (ii) one or more of AFAP1IL2⁺, ASB2⁺, CXCL13⁺, HMOX1⁺, ITM2A⁺, KLRB1⁺, PDLIM4⁺, TIGIT⁺, LTB⁻, LYAR⁻, RGCC⁻, and S100A10⁻;
(b) CD4⁺ and one or more of BATF⁺, CD247⁺, CXCL13⁺, DNPH1⁺, DUSP4⁺, GYPC⁺, IFITM1⁺, IGFLR1⁺, ITM2A⁺, KLRB1⁺, LIMS1⁺, NMB⁺, NR3C1⁺, SH2D1A⁺, SPOCK2⁺, SUPT3H⁺, TNFRSF18⁺, CCL5⁻, CD52⁻, GSTP1⁻, JUN⁻, LGALS1⁻, LTB⁻, LYAR⁻, PLP2⁻, RGCC⁻, S100A10⁻, VIM⁻, and ZFP36⁻;
(c) CD8⁺ and one or more of AFAP1IL2⁺, ALOX5AP⁺, ARHGAP9⁺, ASB2⁺, CARD16⁺, CD3G⁺, CD8A⁺, CD8B⁺, CLIC3⁺, CTSW⁺, CXCL13⁺, CXCR6⁺, GALNT2⁺, GZMB⁺, HLA-DPA1⁺, HLA-DPB1⁺, HLA-DRB1⁺, HLA-DRB5⁺, HMGN3⁺, HMOX1⁺, ITGAE⁺, ITM2A⁺, KLRB1⁺, MPST⁺, NAP1L4⁺, NELL2⁺, NSMCE1⁺, PDLIM4⁺, PTMS⁺, RAB27A⁺, RARRES3⁺, RBPJ⁺, ANXA1⁻, EEF1B2⁻, EMP3⁻, IL7R⁻, LGALS3⁻, LTB⁻, LYAR⁻, RGCC⁻, RPL36A⁻, and S100A10⁻;
(d) CD8⁺ and one or more of CD39⁺, CD74⁺, CD103⁺, CD106⁺, CD137⁺, HLA-DR⁺, TIGIT⁺, CCR7⁻, CD8A⁻, CD16⁻, CD45RA⁻, CD62L⁻ and IL7R⁻;
(e) one or more of ABI3⁺, AC243960.1⁺, ACP5⁺, ADGRG1⁺, AHI1⁺, ASB2⁺, BST2⁺, CARS⁺, CCL4⁺, CD27⁺, CD2BP2⁺, CD82⁺, CTSW⁺, CXCL13⁺, CXCR6⁺, DUSP4⁺, ENTPD1⁺, GALNT2⁺, GATA3⁺, GPR25⁺, GZMB⁺, HDLBP⁺, HLA-DPA1⁺, HLA-DRB1⁺, HMOX1⁺, ID2⁺, IGFLR1⁺, ITGAL⁺, LINC01871⁺, LINC01943⁺, MIS18BP1⁺, MPST⁺, NCF4⁺, NSMCE1⁺, PCED1B⁺, PDCD1⁺, PHPT1⁺, PLEKHF1⁺, PRF1⁺, PTMS⁺, SLC1A4⁺, SLF1⁺, SMC4⁺, SUPT3H⁺, TNFRSF18⁺, TOX⁺, TRAF3IP3⁺, and YPEL2⁺;
(f) CD4⁺ and one or more of ADI1⁺, AHI1⁺, ARID5B⁺, BATF⁺, CMTM7⁺, CPM⁺, CXCL13⁺, CYTH1⁺, ETV7⁺, FABP5⁺, FBLN7⁺, FKBP5⁺, GRAMD1A⁺, HIF1A⁺, IL6ST⁺, ITGA4⁺, ITK⁺, JAK3⁺, KLRB1⁺, LEF1⁺, LIMS1⁺, MAF⁺, MAL⁺, MIR4435-2HG⁺, MYL6B⁺, NAP1L4⁺, NMB⁺, NR3C1⁺, PASK⁺, PGM2L1⁺, PIM2⁺, PPP1CC⁺, SESN3⁺, SH2D1A⁺, SOCS1⁺, STAT1⁺, SYNE2⁺, TBC1D4⁺, TLK1⁺, TMEM123⁺, TMEM70⁺, TNIK⁺, TOX⁺, TSHZ2⁺, UCP2⁺, VOPP1⁺, and YPEL2⁺;
(g) CD8⁺ and one or more of AC243829.4⁺, ACP5⁺, APOBEC3C⁺, APOBEC3G⁺, CCL3⁺, CCL4⁺, CCL4L2⁺, CCL5⁺, CD27⁺, CD8A⁺, CD8B⁺, CST7⁺, CTSW⁺, CXCL13⁺, DUSP4⁺, ENTPD1⁺, FABP5⁺, GALNT2⁺, GNLY⁺, GZMA⁺, GZMB⁺, GZMH⁺, GZMK⁺, HAVCR2⁺, HCST⁺, HLA-DMA⁺, HLA-DPA1⁺, HLA-DPB1⁺, HLA-DRA⁺, HLA-DRB1⁺, HLA-DRB5⁺, HMOX1⁺, IFNG⁺, IGFLR1⁺, ITGAL⁺, JAML⁺, LINC01871⁺, LYST⁺, MIR155HG⁺, NKG7⁺, PLEKHF1⁺, PRF1⁺, PTMS⁺, RGS1⁺, SLF1⁺, SMC4⁺, SUPT3H⁺, TIGIT⁺, and TOX⁺;
(h) one or more of AHI1⁺, CXCL13⁺, FABP5⁺, NAP1L4⁺, ORMDL3⁺, PPP1R16B⁺, SH2D1A⁺, TIGIT⁺, and TOX⁺; or
(i) one or more of TIGIT⁺, CD39⁺, and PD-1⁺.

4. The method of claim 1, wherein the gene expression profile comprises TIGIT+.

5. The method of claim 1, wherein the gene expression profile comprises CXCL13⁺.

6. The method of claim 1, wherein the gene expression profile comprises CD8⁺ and CXCL13⁺.

7. The method of claim 1, wherein the gene expression profile comprises CD4⁺ and CXCL13⁺.

8. The method of claim 1, wherein the gene expression profile comprises CD8⁺, TIGIT⁺, and one or both of CD39⁺ and PD-1⁺.

9. The method of claim 1, wherein the gene expression profile comprises CD8⁺, TIGIT⁺, CD39⁺, and PD-1⁺.

10. The method of claim 1, wherein the gene expression profile comprises CD8⁺, CXCL13⁺, and one or more of CD39⁺, TIGIT⁺, and PD-1⁺.

11. The method of claim 1, wherein the gene expression profile comprises CD8⁺, CXCL13⁺, CD39⁺, TIGIT⁺, and PD-1⁺.

12. The method of claim 1, wherein the gene expression profile comprises CD4⁺, CXCL13⁺, and one or more of CD39⁺, TIGIT⁺, and PD-1⁻.

13. The method of claim 1, wherein the gene expression profile comprises CD4⁺, CXCL13⁺, CD39⁺, TIGIT⁺, and PD-1⁻.

14. The method of claim 1, wherein selecting the isolated T cells which have a gene expression profile comprises:
(i) detecting the presence of protein(s) encoded by positively expressed gene(s) of the gene expression profile;
(ii) detecting the absence of protein(s) encoded by gene(s) that are negative for expression in the gene expression profile;
(iii) measuring the quantity of protein(s) encoded by gene(s) that are negative for expression in the gene expression profile; and/or
(iv) measuring the quantity of protein(s) encoded by gene(s) that are positive for expression in the gene expression profile.

15. The method of claim 1, wherein selecting the isolated T cells which have a gene expression profile comprises:
(i) detecting the presence of RNA encoded by positively expressed gene(s) of the gene expression profile;
(ii) detecting the absence of RNA encoded by gene(s) that are negative for expression in the gene expression profile;
(iii) measuring the quantity of RNA encoded by gene(s) that are negative for expression in the gene expression profile; and/or
(iv) measuring the quantity of RNA encoded by gene(s) that are positive for expression in the gene expression profile.

16. The method of claim 1, wherein selecting the isolated T cells which have a gene expression profile comprises carrying out one or more single cell dimensional reduction methods.

17. The method of claim 1, wherein selecting the isolated T cells which have a gene expression profile comprises carrying out Cellular Indexing of Transcriptomes and Epitopes by Sequencing (CITE-Seq) analysis.

18. The method of claim 1, wherein selecting the isolated T cells which have a gene expression profile comprises carrying out single cell transcriptome analysis.

19. The method of claim 1, wherein selecting the isolated T cells which have the gene expression profile comprises detecting cell surface expression of the one or more genes in the gene expression profile.

20. The method of claim 1, wherein the gene expression profile of (d) further comprises one or both of PD-1⁺ and TIM-3⁺.

21. The method of claim 1, wherein the gene expression profile of (e) or (g) further comprises LAG3⁺.

22. The method of claim 1, wherein the cancer-associated viral antigen is a human papillomavirus (HPV) antigen.

23. A method of preparing a population of cells that express a TCR, or an antigen-binding portion thereof, having antigenic specificity for a target antigen, the method comprising:
isolating a TCR, or an antigen-binding portion thereof, according to the method of claim 2, and
introducing a nucleotide sequence encoding the isolated TCR, or the antigen-binding portion thereof, into peripheral blood mononuclear cells (PBMC) to obtain cells that express the TCR, or the antigen-binding portion thereof.

24. A method of preparing a pooled population of cells that express a TCR, or an antigen-binding portion thereof, having antigenic specificity for a target antigen, the method comprising:
(a) preparing an enriched population of T cells having antigenic specificity for the target antigen according to the method of claim 1;
(b) sorting the T cells in the enriched population into separate single T cell samples;
(c) sequencing TCR complementarity determining regions 3 (CDR3) in the separate single T cell samples;
(d) pairing an alpha chain variable region comprising a CDR3 with a beta chain variable region comprising a CDR3 encoded by the nucleic acid of the separate single T cell samples;
(e) introducing a nucleotide sequence encoding the paired alpha chain variable region and beta chain variable region into peripheral blood mononuclear cells (PBMC) and expressing the paired alpha chain variable region and beta chain variable region by the PBMC; and
(f) carrying out (c), (d), and (e) for a plurality of the separate single T cell samples of the enriched population of T cells having antigenic specificity for the target antigen prepared according to (a), thereby providing a pooled population of cells that express a TCR, or an antigen-binding portion thereof, having antigenic specificity for a target antigen.

25. The method of claim 23, further comprising expanding the numbers of PBMC that express the TCR, or the antigen-binding portion thereof.

26. A TCR, or an antigen-binding portion thereof, isolated according to the method of claim 2.

27. An isolated population of cells prepared according to the method of claim 1.

28. A pharmaceutical composition comprising the isolated population of cells of claim 27 and a pharmaceutically acceptable carrier.

29-30. (canceled)

31. A method of treating or preventing a condition in a mammal, the method comprising:
preparing an enriched population of T cells having antigenic specificity for a target antigen according to the method of claim 1; and
administering the enriched population of T cells to the mammal in an amount effective to treat or prevent the condition in the mammal, wherein the condition is cancer or a viral condition.
